(19) United States
(12) Patent Application Publication
(10) Pub. No.: US 2011/0097332 A1
(43) Pub. Date: Apr. 28, 2011

(54) Composition and Method of Use for HCV Immunization

(76) Inventor: Martina Buck, Del Mar, CA (US)

(21) Appl. No.: 12/672,656

(22) PCT Filed: Aug. 6, 2008
(86) PCT No.: PCT/US08/72368

§ 371 Date: Feb. 8, 2010

Related U.S. Application Data
(60) Provisional application No. 60/954,149, filed on Aug. 6, 2007.

(51) Int. Cl. A61K 39/395; C12N 9/12; C07K 16/10; A61K 39/29; A61P 37/04; A61P 31/14

(52) U.S. Cl. 424/139.1; 435/194; 530/389.4; 424/189.1

(57) ABSTRACT

Isolated HCY E2 kinase phospho-peptides that contain one or more immunogenic fragments of a HCV E2 kinase motif and antibodies which are cross-reactive with the isolated HCV E2 kinase phospho-peptides are provided. Also disclosed are pharmaceutical compositions and/or methods to passively and/or actively immunize against HCV using the isolated HCY E2 kinase phospho-peptides and antibodies.

### REFERENCE TO RELATED APPLICATIONS

This application claims priority to U.S. Provisional Application Ser. No. 60/954,149, entitled “Composition and Method of Use for HCV Immunization,” filed Aug. 6, 2007, the entire content of which is incorporated by reference herewith.

### STATEMENT OF GOVERNMENT INTEREST

This invention was made with government support. As such, the U.S. Government may have certain rights in this invention.

### FIELD OF THE INVENTION

The present invention relates to an immunization therapy for hepatitis C virus (HCV). More particularly, the present invention relates to the development and use of antibodies for passive and/or active immunization against HCV.

### BACKGROUND OF THE INVENTION

Hepatitis C virus (HCV) induces an acute illness and, in over 50% of the infected individuals, will develop into chronic hepatitis. Infected individuals are also at risk of developing hepatocellular carcinoma (HCC) and/or cirrhosis. The global prevalence of chronic HCV is 3% of the population, with approximately 2 new cases per 100,000 persons annually. At present, the cellular mechanisms of HCV infection are not known, and there is no treatment that the majority of patients with HCV respond to. The current therapeutic approach for treating HCV is interferon or interferon plus ribavirin, which is currently the only treatment for HCV infection. These therapies have had, overall, positive effects (approximately a 50% response rate) but there are also serious side effects associated with these therapies. The current treatment also does not eradicate the virus.

The annual global death from liver cirrhosis is approximately 800,000, and there is no available treatment. Excessive tissue repair in chronic liver diseases induced by viral, toxic, immunologic, and metabolic disorders, results in the deposition of scar tissue and the development of cirrhosis. Quiescent hepatic stellate cells produce negligible amounts of extracellular matrix proteins (ECM), but after their activation, these cells develop a myofibroblastic phenotype, proliferate and become the main contributors of ECM, resulting in further development of liver fibrosis and cirrhosis.

Hepatocellular carcinoma (HCC) is the most common primary liver cancer. Approximately, 500,000 new cases of HCC occur worldwide each year (6; 55). In China and sub-Saharan Africa, HCC is the most important cause of cancer-related mortality, while in the USA there are 15,000 new cases of HCC each year (55). The vast majority of HCC develop in patients with chronic liver disease and cirrhosis. The principal causes of cirrhosis leading to HCC include viral hepatitis, alcoholic and non-alcoholic steatohepatitis (NASH), and genetic disorders (6).

In the USA, Europe and Japan, the main cause of HCC is chronic Hepatitis C viral (HCV) infection (6; 16; 20). The rising frequency of HCC in the USA has been attributed to the epidemic of HCV that occurred in the 1960's to 1980's (18; 19). Once liver cirrhosis is established in hosts infected with HCV, HCC develops at a yearly rate of 2-7%, the higher rates being characteristic in Japan (6; 35). Therefore, knowledge about how HCC develops in chronic HCV infection is urgently required in order to prevent the occurrence of this malignancy.

HCC is a highly fatal cancer with a median survival time from the time of diagnosis of 8 months (7). Unfortunately, the only potential curative therapies are resection and liver transplantation. However, only a minority of patients with HCC is eligible or has access to these treatments (6; 7; 45).

The risk for HCC is increased ˜30-fold among patients with chronic HCV infections (5) (6), and the risk is synergistic with alcohol use and type 2 diabetes (28). Only 15% of these patients are treated in the USA due to exclusion criteria secondary to side effects of PEG-Interferon and ribavirin (19). Moreover, among those treated only ˜50% achieve a sustained virological response (56). Thus, only <10% of all HCV patients in the USA achieves a sustained virological response. Further, these patients are at risk of reactivating the infection since the HCV remains, albeit at low concentrations, in blood, in mononuclear cells/macrophages and within the liver (24).

The molecular mechanism by which HCV results in the development of HCC remains unclear (5; 6). Although, valuable information about HCV-induced HCC have been obtained in transgenic mice expressing HCV core (47), no useful small animal models of HCV-induced liver carcinogenesis exists (55). Although HCV core and NS5A proteins have been incriminated in the pathogenesis of HCV-induced HCC (5; 40; 56), these mechanisms remain controversial. Therefore, the mechanisms by which HCV induces HCC have not been established (21)(56) (FIG. 1).

An estimated 3% of the world's population has been exposed to HCV (1) and about 70% of these individuals develop a chronic infection, which may include fibrosis, cirrhosis, and hepatocellular carcinoma (2; 3; 5). However, the mechanisms involved in the HCV cell entry, trafficking, viral assembly, and exit are poorly understood. E2 has been shown to dimerize with E1, and associate with the CD 81 receptor (52) and the LDL receptor (64), although neither association has proven to be the cellular entry mechanism for HCV in humans. The role of E2 in human hepatocytes remains to be characterized.

HCV is a Hepacivirus, from the family Flaviviridae (43), which is comprised of three genera of small-enveloped positive-strand RNA viruses (59). The HCV 9.6 kb genome consists of a single open reading frame (ORF) flanked by 5′ and 3′ nontranslated regions (NTR) (4). The HCV 5′ NTR contains an internal ribosome entry site (IRES), mediating cap-independent translation of the ORF of ˜3,011 amino acids. The resulting polyprotein is processed into 10 proteins. Host signal peptidase cleavages within the N-terminal portion of the polyprotein generate the structural proteins core (C), E1, and E2 as well as the nonstructural proteins (54) (FIG. 2).

The role of E2 in human hepatocytes is poorly understood. Upon examination of the secondary amino acid structure of E2, residues that match those in the catalytic loop of cyclin dependent kinases (CDKs), MAP kinases, GSK, and cdc-like kinases (CMGC) were found (40). These conserved amino acids appear to be closer to the CDKs, which are known to associate with cyclins, with a 43% homology in this region (FIG. 3).

Indeed, HCV E2 was found to be associated with cyclin G (FIG. 4) and has similar amino acid motifs, to that of cyclin G associated kinase (GAK) (FIG. 5). GAK was cloned through its ability to bind to cyclin G (39), and is also known as auxilin 2 due to its homology to auxilin. GAK has been shown to be a master regulator of clathrin-mediated cell trafficking (25; 73) and receptor signaling and function (74). HCV E2 was also found to be homology to the kinase region of GAK, also a member of cyclin dependent kinases (CDKs), MAP kinases, GSK, and cdc-like kinases (CMGC) (40). In its regulation of receptor endocytosis, GAK was proven to be a kinase that phosphorylates the AP50 subunit of adaptor protein-2 (AP2) (50) (FIG. 6). It was further found that HCV E2 would be able to control clathrin-mediated endocytosis through phosphorylation of AP50.

The AP2 complex controls clathrin-mediated endocytosis by providing a bridge between receptors' cargo domain (ΦxxY) (FIG. 6) and the clathrin coat. This occurs through binding of the AP50 (μ2) subunit of AP2 to both the receptors' cargo domain and the clathrin βsubunit (57) (FIG. 7). This binding has been found to be important as clathrin coated pits and transferrin receptor endocytosis are inhibited in AP2 depleted cells (48). The binding of AP50 to receptors requires its phosphorylation (FIG. 6). In addition, the auxilin homologue of C. elegans is necessary for receptor mediated endocytosis (25) and the Aux1, a yeast homologue, is required for effective vesicle transport (53).

Therefore, it is reported that HCV E2 glycoprotein is a regulator of clathrin mediated trafficking (CMT), cell signaling and function. HCV E2 glycoprotein regulates CMT by phosphorylating the clathrin adaptor protein AP50. This phosphorylation facilitates the binding of AP50 to the sorting signals and provides a bridge between the membrane and the clathrin coated vesicles, thereby controlling endocytosis.

Currently, the intracellular roles of HCV E2 protein are unknown. The HCV entry, trafficking, viral assembly and exit remain poorly understood. There is no immunization therapy for HCV.

Recently, researchers (29), (44) (75) (71) were able to replicate genomic HCV in Huh-7-derived hepatoma cells, with the efficient production of HCV viral particles that were infectious to cultured Huh-7-derived cells (44) (71) (75) and chimpanzees (71). The replicon system may facilitate understanding of the molecular pathways activated by HCV proteins that lead to proliferation of hepatocytes and, eventually, to the development of HCC in patients with chronic HCV infection. In addition, the Huh-7/HCV model allows to introduce mutations directly into the HCV viral genome, specifically mutating selected motifs of the E2 protein and then study the effects of these mutations on the lifecycle of HCV.

Given these factors, there is a need to investigate the intracellular roles of HCV E2 protein for either HCV infection or prevention and/or treatment of HCV infection. Moreover, there is a need to better understand the mechanism of HCV entry, trafficking, viral assembly and exit, and to develop an immunization therapy for HCV.

### SUMMARY OF THE INVENTION

The present invention provides antigens and/or antibodies for HCV immunization therapy. More particularly, the present invention identifies specific domains/motifs of HCV E2 kinase comprising one or more immunogenic fragments, and provides antibodies which are cross-reactive with these specific domains/motifs of HCV E2 protein comprising the immunogenic fragments for passive and active immunization for HCV. In one preferred embodiment, the present invention provides that the HCV E2 glycoprotein is a novel kinase that initiates signal transduction mechanisms modulating the following pathways: 1) clathrin-mediated endocytosis, through a site-specific phosphorylation of the clathrin adaptor protein-50 (AP50), a key regulator of clathrin-mediated receptor endocytosis; and 2) hepatocyte proliferation and liver carcinogenesis through the activation of PI3 Kinase and Akt.

The present invention provides isolated HCV E2 kinase phospho-peptides comprising immunogenic fragments of a HCV E2 kinase motif. In one preferred embodiment, the present invention provides a phospho-peptide map, providing potentially important phosphorylation sites of all of the putative phosphorylation sites of HCV E2 kinase. In yet another preferred embodiment, the present invention also provides all of the mutations of the putative phosphorylation sites of the HCV E2 kinase. All of the putative phosphorylation sites (phosphorylated and unphosphorylated), and mutations of these phosphorylation sites, of the HCV E2 kinase are potential targets to make antibodies against HCV E2 kinase.

Yet, the present invention provides about 20 isolated phospho-peptides comprising immunogenic fragments from the full-length HCV E2 kinase with trypsin cleavage. The isolated HCV E2 phospho-peptide contain one or more phosphorylated amino acid, such as tyrosine (Y). The 20 isolated phospho-peptides and their amino acid sequences are listed in the following table:

| | | SEQ ID |
|---|---|---|
| Name | Amino Acid Sequence | NO. |
| Peptide 1 (1) | 1-E T H V T G G S A R | 1 |
| Peptide 2 (11) | 11-T T A G L V G L L T | 2 |
| | P G A K | |
| Peptide 3 (25) | 25-Q N I Q L I N T N G | 3 |
| | S W H T N S T A L N C | |
| | N E S L N T G W L A G | |
| | L F Y Q H K | |
| Peptide 4 (63) | 63-F N S S G C P E R | 4 |
| Peptide 5 (72) | 72-L A S C R | 5 |
| Peptide 6 (77) | 77-L T D F A Q G W G P I | 6 |
| | S Y A N G S G L D E R | |
| Peptide 7 (99) | 99-P Y C W H Y P P R, | 7 |
| Peptide 8 (108) | 108-S V C G P V Y C F T | 8 |
| | P S P V V V G T T D R | |
| Peptide 9 (129) | 129-S G A P T Y S W G A | 9 |
| | N D T D V F V L N N T R | |
| Peptide 10 (151) | 151-P P L G N W F G C | 10 |
| | T W M N S T G F T K | |
| Peptide 11 (170) | 170-V C G A P P C V I | 11 |
| | G G V G N N T L L C P | |
| | T D C F R | |
| Peptide 12 (196) | 196-H P E A T Y S R | 12 |
| Peptide 13 (204) | 204-C G S G P W I T P R | 13 |
| Peptide 14 (214) | 214-C M V D Y P Y R | 14 |
| Peptide 15 (222) | 222-L W H Y P C T I N Y | 15 |
| | T I F K | |
| Peptide 16 (236) | 236-M Y V G G V E H R | 16 |
| Peptide 17 (245) | 245-L E A A C N W T R | 17 |
| Peptide l8 (254) | 254-S E L S P L L L S T | 18 |
| | T Q W Q V L P C S F T T | |
| | L P A L S T G L I H L H | |
| | Q N I V D Q Y L Y G V G | |
| | S S I A S W A I K | |
| Peptide 19 (309) | 309-W E Y V V L L F | 19 |
| | L L L A D A R | |
| Peptide 20 (324) | 324-V C S C L W M M | 20 |
| | L L I S Q A E A | |

In yet another preferred embodiment, the present invention further provides isolated peptides comprising HCV E2 motifs that containing conserved, polar or non-polar, or exact matched amino acids with other kinases, such as AAK and GAK. In preferred embodiments, the peptides comprise amino acid sequences [symbol] (SEQ ID NO:21), [symbol] (SEQ ID NO:22), [symbol] (SEQ ID NO:23), [symbol] (SEQ ID NO:24), [symbol] (SEQ ID NO:25), [symbol] (SEQ ID NO:26), [symbol] [symbol] [symbol] [symbol] (SEQ ID NO:28), [symbol] [symbol] (SEQ ID NO:29), [symbol] [symbol] [symbol] (SEQ ID NO:30), [symbol] [symbol] (SEQ ID NO:31), [symbol] [symbol] [symbol] (SEQ ID NO:32), immunogenic fragments, or homologs thereof.

In yet another preferred embodiment, the present invention provides antibodies that interact with the unphosphorylated and/or phosphorylated sites of the HCV E2 kinase phospho-peptides. In preferred embodiments, antibodies cross-reactive with the immunogenic fragments of the phosphorylated and/or unphosphorylated motifs of the 20 phospho-peptides presented herewith are also provided. In one preferred embodiment, an antibody E2o to an unphosphorylated motif and an antibody E2p to a phosphorylated motif of the peptide 14 (214) (SEQ ID NO:14) were produced and tested for HCV infection in primary human hepatocytes with genotype 1 patient serum. In yet another preferred embodiment, an antibody to an immunogenic fragment of ELSPLL (SEQ ID NO:33) or repeated immunogenic fragment of LSPLLELSPLLELSPLLELSPLL (SEQ ID NO:34) is generated and tested for HCV immunization.

In yet another preferred embodiment, the present invention provides a vaccine development for HCV immunization therapy. In preferred embodiments, the present invention provides antigens (active vaccine) comprising the isolated HCV E2 phospho-peptides comprising amino acid sequences as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:l7, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, immunogenic fragments, or homologs thereof, for active immunization of HCV. The present invention further provides passive vaccine comprising the antibodies that are cross-reactive with the isolated HCV E2 phospho-peptides.

Furthermore, the present invention provides a pharmaceutical composition, and/or method of use thereof, to passively and/or actively immunize against HCV, comprising administering a subject in need an effective amount of one or more isolated HCV E2 phospho-peptides, or antibodies and/or vaccines developed by the present invention.

### BRIEF DESCRIPTION OF THE DRAWINGS

FIG. 1 illustrates progression of HCC. Hepatic injury caused by any of several factors, (hepatitis B virus, hepatitis C virus, alcohol, and aflatoxin B1) results in necrosis followed by hepatocyte proliferation. Repeated cycles of destruction-regeneration result in chronic liver disease characterized by cirrhosis. Hyperplastic nodules are followed by dysplastic nodules which develop into hepatocellular carcinoma (21).

FIG. 2 illustrates HCV life cycle.

FIG. 3 illustrates homology of CDK and E2 kinase II domain catalytic loops. The CDK consensus (SEQ ID NO:42) is shown compared to the HCV E2 (SEQ ID NO:43). There is a 43% homology, including allowable substitutions. The central K residue is shown (K25R mutation in E2) (SEQ ID NO:44).

FIG. 4 illustrates association between E2 and Cyclin G. In primary mouse hepatocytes, endogenous cyclin G associates with wild type E2 (lane 1), K25R (lane 2), but not with Y228E and Y228F (lanes 3 and 4). Immuno-purification performed with anti-cyclin G antibodies. Immuno-purifications with anti-E2 antibodies show similar results (data not shown).

FIG. 5 illustrates homology between GAK (SEQ ID NO:45) and E2. The proposed 10 mutations of E2 (SEQ ID NO:46) are shown in blue (SEQ ID NO:47). The putative cargo motif is underlined in red.

FIG. 6 illustrates structure of Adaptor Protein Complex (AP50). Unphosphorylated AP50 is in a closed conformation, with no binding to receptors on the plasma membrane. Phosphorylation of AP50 confers an open conformation and the ability to bind to receptors with a cargo domain, increasing endocytosis of these receptors and their ligands (67).

FIG. 7 illustrates structure of AP50/μ2 within the clathrin coated pit. Phosphorylated AP50 forms a bridge between the receptors on the external side of the plasma membrane of the coated pit and the clathrin triskelia that make up the structure of the coated pit. AP50 does this by binding to the clathrin β subunit and the cargo domains of the receptors.

FIG. 8 illustrates association of AP50 and E2 in primary hepatocytes. In primary mouse hepatocytes, endogenous AP50 associates with wild type E2 (lane 1), K25R (lane 2), and Y228E and Y228F (lanes 3 and 4) Immuno-purification studies done with anti-AP50 antibodies. Immuno-purifications performed with antibodies to E2 show similar results (data not shown).

FIG. 9 illustrates that E2 phosphorylates AP50 in a cell-free system. Recombinant wild type E2 phosphorylates AP50 (lane 2) compared to control in the absence of E2 (lane 1). E2 kinase activity was decreased with the K25R (lane 3), Y228E (lane 4), and Y228F (lane 5) mutants.

FIG. 10 illustrates multiple alignments of 7 representative kinases. HCV E2, NCBI P26664, C. elegans AAK, NP_{—}497929, P. faciparum, NP_{—}701816, C. elegans GAK NP_{—}508971, Enterobacteria phage T7, NP_{—}041959, Staphylococcus aureus SaCoaA, 15599475, and Mycobacterium tuberculosis pknH Q11053. HCV E2, C. elegans AAK and GAK, and Plasmodium farciparum GAK are all members of the Ark/Prk family of kinases. The other kinases are compared as non-family members to show the general structural relationship among other non-eukaryotic protein kinases. o nonpolar residues, uppercase letters invariant residues, lowercase letters nearly invariant residues, * polar residues. Residues that the sequences have in common, either polar or nonpolar, or exact matches are highlighted in yellow. Nomenclature taken from Hanks and Hunter (24). FIG. 10A discloses SEQ ID NOS:21 and 48-53, respectively in order of appearance. FIG. 10B disclosed SEQ ID NOS:22 and 54-59, respectively in order of appearance. FIG. 10C discloses SEQ ID NOS:23 and 60-65, respectively in order of appearance. FIG. 10D disclosed SEQ ID NOS:24 and 66-71, respectively in order of appearance. FIGS. 10E-F disclose SEQ ID NOS:72-85, respective in order of appearance. FIG. 10G discloses SEQ ID NOS:27 and 86-91, respectively in order of appearance. FIG. 10H discloses SEQ ID NOS:98-104, respectively in order of appearance. FIG. 10J discloses SEQ ID NOS:30 and 105-110, respectively in order of appearance. FIGS. 10K-L disclose SEQ ID NOS:111-124, respectively in order of appearance.

FIG. 11 illustrates Phylogenic Tree (A) and Diagram (B) of Ark/Prk domains. A. The yeast homologues (pink), the AAk members (green), and the GAK members (yellow), all belong to separate groups. B. The kinase domain is near the amino terminus (red), with a variable length region downstream from the kinase motif. Only two of the proteins have other recognizable homologous domains; J domains (blue) (69).

FIG. 12 illustrates that a non-phosphorylatable mutant AP50 peptide is able to block the phosphorylation of AP50 by recombinant E2 in vitro. Addition of the mutant AP50 peptide to the in vitro phosphorylation assay blocks AP50 phosphorylation by E2. The auto-phosphorylation of E2 is unaffected by the AP50 peptide, demonstrating that the auto-phosphorylation of E2 is independent of AP50 association. The inhibition of phosphorylation of AP50 by the peptide indicates that its phosphorylation is dependent upon association with E2.

FIG. 13 illustrates that HCV E2 is co-localized with AP50 in E2-transfected primary mouse hepatocytes and liver from HCV-infected patients. Antibodies specific to E2 and AP50 were used together with secondary fluorochromes to visualize E2 in green and AP50 in red. The yellow fluorescence in the merge field indicates a co-localization of the two proteins. There is only a co-localization between AP50 and HCV E2 in the samples containing E2.

FIG. 14 illustrates that E2 associates with AP50 in the liver of infected patients. E2 and AP50 immuno-blots from AP50 immuno-purification in control lane 1 and HCV infected patients lanes 2 and 3. Immuno-purifications were performed with antibodies to AP50. Immuno-purifications done with antibodies to E2 showed similar results (data not shown).

FIG. 15 illustrates that AP50 is phosphorylated in E2-transfected primary mouse hepatocytes and liver from HCV-infected patients. The phosphorylation of AP50 was measured with an antibody specific to the threonine 156 phospho-acceptor of APR50. It was visualized in red by a secondary antibody linked to a red emitting Q dot (Molecular Probes). The phosphorylation is only significantly increased above background in the samples containing E2.

FIG. 16 illustrates that HCV mRNA is comparable at 48 hours in HCV-infected primary human hepatocytes to that in HCV-infected liver samples. There is consistent production of HCV RNA, by RT-PCR, of genotype 1 (closed bars), genotype 3 (open bars), and genotype 4 (striped bars) for up to 3 weeks.

FIG. 17 illustrates an amplification of the HCV E2 protein in HCV-infected primary human hepatocytes. HCV E2 protein was immuno-purified from HCV-infected primary human hepatocytes and subjected to western analysis. Genotypes 1 (lane 1), 2 (lane 2), 3 (lane 3), and 4 (lane 4) were increased exponentially when compared to infected cells at time zero.

FIG. 18 illustrates that E2 induces DNA replication. In primary mouse hepatocytes, control thymidine incorporation (lane 1) was increased by TGFαEGF and E2 (lanes 2, 3 and 4), but not by E2 mutant K25R, Y228E or Y228F (lanes 5, 6 and 7).

FIG. 19 illustrates that PCNA, an indicator of proliferation, is found in liver from HCV Infected patients. Immuno-staining for nuclear PCNA (red) is apparent in HCV biopsies (lower middle panel) indicating proliferation, while negligible in control sample (middle top panel).

FIG. 20 illustrates that HCV E2 increases the endocytosis of the transferrin receptor. [¹²⁵I] internalization studies of transferrin show that E2 increases the endocytosis of the transferrin receptor (magenta line), in E2 transfected primary mouse hepatocytes when compared to control (blue line).

FIG. 21 illustrates regulation of Akt. Akt is translocated to the membrane upon PIP₃ production from PIP₂ by PI3K (p110 subunit). Akt is phosphorylated by PDK1 on Thr308 and by PDK2 on Ser473. Phosphorylation on both sites leads to Akt activation (70).

FIG. 22 illustrates that HCV E2 increases PIP₂ and leads to the activation of the PI3K signal transduction cascade. A. PIP₂ is immuno-purified and shown to be increased with E2 transfection, lane 2, above control lane 1, and K25R, Y228E/Y228F in lanes 3, 4, and 5 are also increased. B. PI3K is shown to be increased and activated with E2 transfection, lane 2 over control lane 1. K25R is also able to stimulate PI3K, lane 3, while Y228E/F are not, lanes 4 and 5. C. PDK1 is also increased and activated by E2, lane 2 above control, lane 1. The K25R, Y228E/F mutants are also able to activate PDK1 although not as well as E2, lanes 3, 4, and 5. D. Akt is also increased and activated by E2, lane 2, when compared to control, lane 1. K25R is unable to activate Akt, lane 3, while Y228E/F can, lanes 4 and 5. E. BAD is phosphorylated in response to E2, lane 2, when compared to control, lane 1. K25R, and Y228E/F mutants lead to a lesser phosphorylation of BAD, lanes 3, 4 and 5. Immuno-purifications were performed with antibodies to PIP2, PI3K, PDK1, Akt, and BAD.

FIG. 23 illustrates that active Akt is increased in the livers of HCV infected patients. In immuno-purified Akt from the liver of HCV infected patients, phosphorylated or active Akt is increased as measured by immuno-blots, lane 2 and 3, when compared to uninfected, control liver samples, lane 1. Immuno-purifications were performed with antibodies to Akt.

FIG. 24 illustrates that the AP50 peptide was cell permeable and co-localized with HCV E2 in HCV-infected primary human hepatocytes. Immuno-staining with antibodies specific to were used to visualize E2. E2 is shown with a secondary antibody conjugated to TR (red) and AP50 is shown by its FITC (green) tag. Yellow, in the merge field is the co-localization of E2 and the peptide, NH2-QGEVQRRRQRRKKRGYGGGG-FITC (SEQ ID NO:36).

FIG. 25 illustrates that treatment of HCV-infected primary human hepatocyte cultures with the AP50 peptide inhibits HCV replication of genotypes 1, 3, and 4. It was found that addition of the AP50 peptide at the time of HCV infection decreases the HCV RNA produced from HCV genotypes 1, 3, and 4 in primary human hepatocytes as measured by RT-PCR.

FIG. 26 illustrates a decreased toxicity in HCV-infected primary human hepatocytes with the addition of the AP50 peptide. Upon addition of a AP50 peptide to the media at 72 hours post HCV infection (after an aliquot of the media is taken for LDH assays), the LDH levels began to decrease indicating a decrease in hepatocyte toxicity. In the presence of a AP50 peptide, the toxicity continued to decrease until the termination of the experiment at 120 hours when the toxicity was comparable to that of uninfected cells.

FIG. 27 illustrates putative phosphorylation sites and possible phosphorylation sites of HCV E2 kinase. Trypsin cleavage generates 20 peptides from the full-length E2 kinase that contain phosphorylated amino acid.

FIG. 28 illustrates a predictive 2-D phospho-map of E2 phospho-peptides generated from trypsin cleavage. The peptides are mapped according to their charge along the X-axis and their hydrophobicity along the y-axis.

FIG. 29 illustrates an antibody blockade of HCV infection in the primary human hepatocytes with genotype 1 patient serum. Antibodies to the Y and surrounding motif of peptide 214 were generated. E2o antibody is to an unphosphorylated motif and antibody E2p is to a phosphorylated motif.

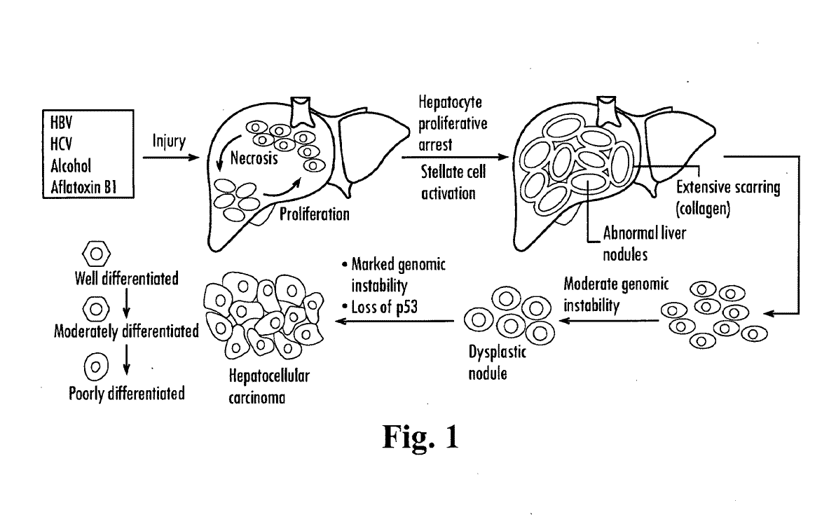

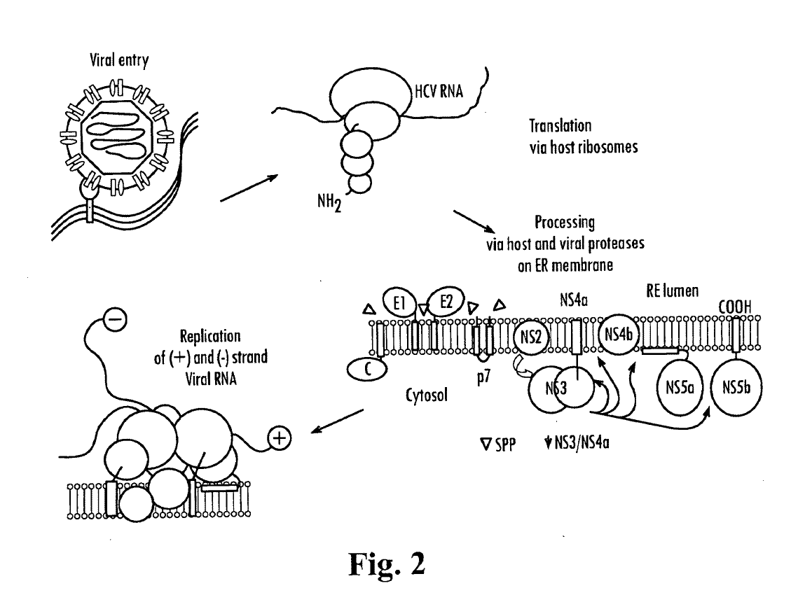

### DETAILED DESCRIPTION OF THE INVENTION

The present invention provides isolated phospho-peptides of HCV E2 kinase comprising an immunogenic fragment of a HCV E2 kinase motif. In particular, the present invention provides about 20 isolated phospho-peptides generated by trypsin cleavage from the full length HCV E2 kinase, having amino acid sequences as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, mutants, immunogenic fragments, analogs, or homologs thereof. The present invention also provides HCV E2 motifs comprising amino acid sequences as set forth in SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, mutants, immunogenic fragments, analogs, or homologs thereof.

As used herein, the term “peptide” refers to a chain of at least three amino acids joined by peptide bonds. The term “peptide” and “protein” are use interchangeably. The chain may be linear, branched, circular, or combinations thereof. As used herein, the term “analogs” refers to two amino acids that have the same or similar function, but that have evolved separately in unrelated organisms. As used herein, the term “analog” further refers to a structural derivative of a parent compound that often differs from it by a single element. As used herein, the term “analog” also refers to any peptide modifications known to the art, including but are not limited to changing the side chain of one or more amino acids or replacing one or more amino acid with any non-amino acids.

In certain embodiments the peptides and analogs of the present invention are isolated or purified. Protein purification techniques are well known in the art. These techniques involve, at one level, the homogenization and crude fractionation of the cells, tissue or organ to peptide and non-peptide fractions. The peptides of the present invention may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. A particularly efficient method of purifying peptides is fast protein liquid chromatography (FPLC) or even HPLC.

An isolated peptide is intended to refer to a peptide/protein that is purified to any degree relative to its naturally-occurring state. Therefore, an isolated or purified peptide refers to a peptide free from at least some of the environment in which it may naturally occur. Generally, “purified” will refer to a peptide composition that has been subjected to fractionation to remove various other components, and which composition substantially retains its expressed biological activity. Where the term “substantially purified” is used, this designation will refer to a composition in which the peptide forms the major component of the composition, such as constituting about 50%, about 60%, about 70%, about 80%, about 90%, about 95%, or more of the peptides in the composition.

Various methods for quantifying the degree of purification of the peptide are known in the art. These include, for example, determining the specific activity of an active fraction, or assessing the amount of peptides within a fraction by SDS/PAGE analysis. Various techniques suitable for use in peptide/protein purification are well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the peptides and their analogs always be provided in their most purified state. Indeed, it is contemplated that less substantially purified products will have utility in certain embodiments. Partial purification may be accomplished by using fewer purification steps in combination, or by utilizing different forms of the same general purification scheme. Methods exhibiting a lower degree of relative purification may have advantages in total recovery of protein product, or in maintaining the activity of an expressed protein. The invention contemplates compositions comprising the peptides and a pharmaceutically acceptable carrier.

In certain embodiments, the peptides and their analogs of the present invention may be attached to imaging agents including but are not limited to fluorescent, and/or radioisotopes including but are not limited to ¹²⁵I, for imaging, diagnosis and/or therapeutic purposes. Many appropriate imaging agents and radioisotopes are known in the art, as are methods for their attachment to the peptides.

The present invention also provides isolated nucleotides encoding the aforementioned phospho-peptides of HCV E2 kinase that contain an immunogenic fragment of a HCV E2 motif. In one of the preferred embodiments, the present invention provides an isolated nucleotide encoding a peptide comprising a phospho-peptide generated by trypsin cleavage from the full length HCV E2 kinase, having an amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, mutants, immunogenic fragments, analogs, or homologs thereof. In yet another preferred embodiment, the present invention provides an isolated nucleotide encoding a peptide comprising a HCV E2 motif comprising an amino acid sequence as set forth in SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, or mutants, immunogenic fragments, analog, or homologs thereof.

As used herein, the “nucleic acids” or “nucleotides” may be derived from genomic DNA, complementary DNA (cDNA) or synthetic DNA. The term “nucleic acid” or “nucleotide” also refer to RNA or DNA that is linear or branched, single or double stranded, chemically modified, or a RNA/DNA hybrid thereof. It is contemplated that a nucleic acid within the scope of the present invention may comprise 3-100 or more nucleotide residues in length, preferably, 9-45 nucleotide residues in length, most preferably, 15-24 nucleotide residues in length. Where incorporation into an expression vector is desired, the nucleic acid may also comprise a natural intron or an intron derived from another gene. Less common bases, such as inosine, 5-methylcytosine, 6-methyladenine, hypoxanthine, and others can also be used.

An “isolated” nucleic acid molecule is one that is substantially separated from other nucleic acid molecules which are present in the natural source of the nucleic acid (i.e., sequences encoding other polypeptides). Preferably, an “isolated” nucleic acid is free of some of the sequences which naturally flank the nucleic acid (i.e., sequences located at the 5′ and 3′ ends of the nucleic acid) in its naturally occurring replicon. For example, a cloned nucleic acid is considered isolated. A nucleic acid is also considered isolated if it has been altered by human intervention, or placed in a locus or location that is not its natural site, or if it is introduced into a cell by agroinfection. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

As used herein, “homologs” are defined herein as two nucleic acids or peptides that have similar, or substantially identical, nucleic acids or amino acid sequences, respectively. The term “homolog” further encompasses nucleic acid molecules that differ from one of the nucleotide sequences due to degeneracy of the genetic code and thus encodes the same amino acid sequences. In one of the preferred embodiments, homologs include allelic variants, orthologs, paralogs, agonists, and antagonists of nucleic acids encoding the peptide, or analogs thereof, of the present invention.

As used herein, the term “orthologs” refers to two nucleic acids from different species, but that have evolved from a common ancestral gene by speciation. Normally, orthologs encode peptides having the same or similar functions. In particular, orthologs of the invention will generally exhibit at least 80-85%, more preferably 85-90% or 90-95%, and most preferably 95%, 96%, 97%, 98%, or even 99% identity, or 100% sequence identity, with all or part of the amino acid sequence of the peptides, or analogs thereof, of the present invention, preferably, SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, mutants, immunogenic fragments, analogs, or homologs thereof. Preferably, the orthologs of the present invention associate with HCV E2 kinase and function as HCV E2 kinase. As also used herein, the term “paralogs” refers to two nucleic acids that are related by duplication within a genome. Paralogs usually have different functions, but these functions may be related (Tatusov et al., 1997, Science 278(5338):631-637).

To determine the percent sequence identity of two amino acid sequences, the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence is occupied by the same amino acid residue as the corresponding position in the other sequence, then the molecules are identical at that position. The same type of comparison can be made between two nucleic acid sequences.

The percent sequence identity between the two sequences is a function of the number of identical positions shared by the sequences (i.e., percent sequence identity=numbers of identical positions/total numbers of positions×100). Preferably, the isolated amino acid homologs included in the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to an entire amino acid sequence shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, mutants, immunogenic fragments, analogs, or homologs thereof.

The determination of the percent sequence identity between two nucleic acid or peptide sequences is well known in the art. For instance, the Vector NTI 6.0 (PC) software package (InforMax, 7600 Wisconsin Ave., Bethesda, Md. 20814) to determine the percent sequence identity between two nucleic acid or peptide sequences can be used. In this method, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

In another aspect, the present invention provides an isolated nucleic acid comprising a nucleotide sequence that hybridizes to the nucleotides encoding the amino acid sequences shown in SEQ ID NO:1, SEQ ID NO:2, SEQ ID NO:3, SEQ ID NO:4, SEQ ID NO;5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO;25, SEQ ID NO:26, SEQ ID NO:27, SEQ ID NO:28, SEQ ID NO:29, SEQ ID NO:30, SEQ ID NO:31. SEQ ID NO:32, SEQ ID NO:33, SEQ ID NO:34, SEQ ID NO:35, mutants, immunogenic fragments, analogs, or homologs thereof, under stringent conditions.

As used herein with regard to hybridization for DNA to a DNA blot, the term “stringent conditions” refers to hybridization overnight at 60° C. in 10× Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10× Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for nucleic acid hybridizations are described in Meinkoth and Wahl, 1984, Anal. Biochem. 138:267-284; Current Protocols in Molecular Biology, Chapter 2, Ausubel et al., eds., Greene Publishing and Wiley-Interscience, New York, 1995; and Tijssen, 1993, Laboratory Techniques in Biochemistry and Molecular Biology: Hybridization with Nucleic Acid Probes, Part I, Chapter 2, Elsevier, New York, 1993.

Using the above-described methods, and others known to those of skill in the art, one of ordinary skill in the art can isolate homologs of the peptides of the present invention comprising the amino acid sequence shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, mutants, immunogenic fragments, analogs, or homologs thereof. One subset of these homologs are allelic variants. As used herein, the term “allelic variant” refers to a nucleotide sequence containing polymorphisms that lead to changes in the amino acid sequences of the peptides of the present invention without altering the functional activities. Such allelic variations can typically result in 1-5% variance in nucleic acids encoding the peptides of the present invention.

In addition, the skilled artisan will further appreciate that changes can be introduced by mutation into a nucleotide sequence that encodes the amino acid sequence of the peptides, or analogs thereof, of the present invention. For example, nucleotide substitutions leading to amino acid substitutions at “non-essential” amino acid residues can be made in a sequence encoding the amino acid sequence of the peptides, or analogs thereof, of the present invention. A “non-essential” amino acid residue is a residue that can be altered without altering the activity of said peptide, whereas an “essential” amino acid residue is required for desired activity of such peptide, such as enhance or facilitate transdermal delivery of any drugs.

In one embodiment, the isolated nucleic acid molecule comprises a nucleotide sequence encoding a peptide, wherein the peptide comprises an amino acid sequence at least about 50% identical to an amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, mutants, immunogenic fragments, analogs, or homologs thereof. Preferably, the peptide encoded by the nucleic acid molecule is at least about 50-60% identical to an amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ED NO:17, SEQ ID NO:18, SEQ ID NO:19, SEQ ED NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, mutants, immunogenic fragments, analogs, or homologs thereof, more preferably at least about 60-70% identical, even more preferably at least about 70-75%, 75-80%, 80-85%, 85-90%, or 90-95% identical, and most preferably at least about 96%, 97%, 98%, or 99% identical to an amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, mutants, immunogenic fragments, analogs, or homologs thereof.

An isolated nucleic acid molecule encoding the peptides of the present invention can be created by introducing one or more nucleotide substitutions, additions, or deletions into a nucleotide encoding the peptide sequence, such that one or more amino acid substitutions, additions, or deletions are introduced into the encoded peptide and/or the side chain of the amino acids constituting the encoded peptides. Mutations can be introduced into the nucleic acid sequence encoding the peptide sequence of the present invention by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain.

Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Following mutagenesis of the nucleic acid sequence encoding the peptides of the present invention, the encoded peptide can be expressed recombinantly and the activity of the peptide can be determined by analyzing its catalyze activity of HCV E2 kinase.

The nucleotides of the present invention may be produced by any means, including genomic preparations, cDNA preparations, in vitro synthesis, RT-PCR, and in vitro or in vivo transcription. It is contemplated that peptides of the present invention, their variations and mutations, or fusion peptides/proteins may be encoded by any nucleic acid sequence that encodes the appropriate amino acid sequence. The design and production of nucleic acids encoding a desired amino acid sequence is well known to those of skill in the art based on standardized codons. In preferred embodiments, the codons selected for encoding each amino acid may be modified to optimize expression of the nucleic acid in the host cell of interest. Codon preferences for various species of host cell are well known in the art.

Any peptides and their analogs comprising the isolated peptides of the present invention can be made by any techniques known to those of skill in the art, including but are not limited to the recombinant expression through standard molecular biological techniques, the conventional peptide/protein purification and isolation methods, and/or the synthetic chemical synthesis methods. The nucleotide and peptide sequences corresponding to various genes may be found at computerized databases known to those of ordinary skill in the art, for instance, the National Center for Biotechnology Information's Genbank and GenPept databases (National Center for Biotechnology information). Alternatively, various commercial preparations of proteins and peptides are known to those of skill in the art.

Because the length of the isolated peptides of the present invention is relatively short, peptides and analogs comprising the amino acid sequences of these isolated peptide inserts can be chemically synthesized in solution or on a solid support in accordance with conventional techniques. Various automatic synthesizers are commercially available and can be used in accordance with known protocols. Short peptide sequences, usually from about 5 up to about 35 to 50 amino acids, can be readily synthesized by such methods. Alternatively, recombinant DNA technology may be employed wherein a nucleotide sequence which encodes a peptide and its analog of the present invention is inserted into an expression vector, transformed or transfected into an appropriate host cell, and cultivated under conditions suitable for expression.

Peptide mimetics may also be used for preparation of the peptides and their analogs of the present invention. Mimetics are peptide-containing molecules that mimic elements of protein secondary structure. A peptide mimetic is expected to permit molecular interactions similar to the natural molecule, and may be used to engineer second generation molecules having many of the natural properties of the peptides, but with altered and even improved characteristics.

The present invention also provides chimeric or fusion peptides that comprise the amino acid sequences of the isolated phospho-peptides of the present invention, as disclosed herein. As used herein, a “chimeric or fusion peptide” comprises the amino acid sequence corresponding to the amino acid sequence of the peptides, or analogs thereof, of the present invention, operatively linked, preferably at the N- or C-terminus, to all or a portion of a second peptide or protein. As used herein, “the second peptide or protein” refer to a peptide or protein having an amino acid sequence which is not substantially identical to the amino acid sequences of the phospho-peptides, analogs, or mutants, thereof, of the present invention, e.g., a peptide or protein that is different from HCV E2 kinase motifs, or analogs thereof, and is derived from the same or a different organism. With respect to the fusion peptide, the term “operatively linked” is intended to indicate that the amino acid of the peptides, or analogs thereof, of the present invention, and the second peptide or protein are fused to each other so that both sequences fulfill the proposed function attributed to the sequence used.

For example, fusions may employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. In preferred embodiments, the fusion proteins of the present invention comprise the peptide and/or analog comprising amino acid sequences of the displayed peptide identified from the in vivo phage display, that is linked to a therapeutic protein or peptide. Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. These examples are not meant to be limiting and it is contemplated that within the scope of the present invention virtually any protein or peptide could be incorporated into a fusion protein comprising the peptides and analogs of the present invention. Furthermore, in certain preferred embodiments, the fusion proteins of the present invention exhibit enhanced transdermal penetration capability as compared to non-fusion proteins or peptides that have not fused with the peptides and analogs, as disclosed herein.

Methods of generating fusion peptides/proteins are well known to those of skill in the art. Such peptides/proteins can be produced, for example, by chemical attachment using bifunctional cross-linking reagents, by de novo synthesis of the complete fusion peptide/protein, or by standard recombinant DNA techniques that involve attachment of a DNA sequence encoding the peptides of present invention, as disclosed herein, to a DNA sequence encoding the second peptide or protein, followed by expression of the intact fusion peptide/protein using. For example, DNA fragments coding for the peptide sequences of the phospho-peptides, or analogs thereof, of the present invention, are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and re-amplified to generate a chimeric gene sequence (See, for example, Current Protocols in Molecular Biology, Eds. Ausubel et al., 1992, John Wiley & Sons). Moreover, many expression vectors are commercially available that already encode a fusion moiety (e.g., a GST polypeptide).

The nucleic acids encoding phospho-peptides, analogs, or mutants thereof, of the present invention can be cloned into such an expression vector such that the fusion moiety is linked in-frame to these nucleic acids encoding phospho-peptides, or analogs or mutants thereof, of the present invention. As used herein, a term “vector/virus” refers to a carrier molecule that carries and delivers the “normal” therapeutic gene to the patient's target cells. Because viruses have evolved a way of encapsulating and delivering their genes to human cells in a pathogenic manner, most common vectors for gene therapy are viruses that have been genetically altered to carry the normal human DNA. As used herein, the viruses/vectors for gene therapy include retroviruses, adenoviruses, adeno-associated viruses, and herpes simplex viruses. The term “retrovirus” refers to a class of viruses that can create double-stranded DNA copies of their RNA genomes, which can be further integrated into the chromosomes of host cells, for example, Human immunodeficiency virus (HIV) is a retrovirus. The term “adenovirus” refers to a class of viruses with double-stranded DNA genomes that cause respiratory, intestinal, and eye infections in human, for instance, the virus that cause the common cold is an adenovirus. The term “adeno-associated virus” refers to a class of small, single-stranded DNA viruses that can insert their genetic material at a specific site on chromosome 19. The term “herpes simplex viruses” refers to a class of double-stranded DNA viruses that infect a particular cell type, neurons. Herpes simplex virus type 1 is a common human pathogen that causes cold sores.

The present invention further provides antigens, vaccines, and/or antibodies generated from, and/or comprising the HCV E2 motifs comprising conservative, polar or non-polar, or exact matched amino acids, or to the phosphorylated and/or unphosphorylated motifs of the phospho-peptides of HCV E2 kinase of the present invention for passive and active immunization for HCV. In preferred embodiments, vaccines and antibodies are generated from and/or comprising the phospho-peptides of a HCV E2 motif comprising an amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ED NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, mutants, immunogenic fragments, analogs, or homologs thereof. In one of the preferred embodiments, antibodies to the phosphorylated site, such as tyrosine (Y) (E2p), and to the unphosphorylated motif (E2o), of peptide 14 (also called as “the '214 peptide”, SEQ ID NO:14) were generated and tested for their ability of blockage of HCV infection. In another preferred embodiment, antibodies to SEQ ID NO:33 or SEQ ID NO:34 were generated.

As used herein, the term “antibody” includes complete antibodies, as well as fragments thereof (e.g., F(ab′)2, Fab, etc.) and modified antibodies produced therefrom (e.g., antibodies modified through chemical, biochemical, or recombinant DNA methodologies), with the proviso that the antibody fragments and modified antibodies retain antigen binding characteristics sufficiently similar to the starting antibody so as to provide for specific detection of antigen.

Antibodies may be prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

As used herein, the term “vaccine” refers to a product that produces immunity therefore protecting the body from the disease. Vaccines that comprise a suspension of attenuated or killed microorganism (e.g. bacterial, viruses, or) are administered for the prevention, amelioration or treatment of infectious diseases. In preferred embodiments, the present invention provides HCV vaccines generated from, and/or comprising the isolated phospho-peptide of the HCV E2 kinase motifs, as provided herewith, mutants or analogs thereof, of the present invention.

The present invention further provides a pharmaceutical composition for treating HCV infections comprising the isolated phospho-peptides of HCV E2 kinase that contain phosphorylated amino acids, mutants, or analogs thereof, of the present invention, and any pharmaceutically acceptable excipients. The present invention also provides a pharmaceutical composition for HCV immunization therapy comprising vaccines or antibodies generated from and/or comprising the isolated phospho-peptides of HCV E2 kinase that contain phosphorylated amino acids, mutants, or analogs thereof, of the present invention, and any pharmaceutically acceptable excipients Pharmaceutically acceptable excipients are well known in the art, and have been amply described in variety of publications, including, for example, “Remington: The Science and Practice of Pharmacy”, 19^{th} Ed. (1995).

The present invention further comprises methods for preventing or treating HCV infection comprising administering to a subject at need an effective amount of pharmaceutical composition comprising the isolated phospho-peptides, mutants, or analogs thereof, of the present invention. In preferred embodiments, the isolated phospho-peptides, mutants, or analogs thereof, can be used as a therapeutic agent for treating HCV infection. In yet other preferred embodiments, the present invention provides a method for HCV immunization therapy comprising administering to a subject at need an effective amount of a vaccine or antibody generated from and/or comprising the isolated phospho-peptides, mutants, or analogs thereof, of the present invention, or pharmaceutical composition comprising the forementioned vaccines and/or antibodies of the present invention.

As used herein, the term “therapeutic agent,” “or “drug” is used interchangeably to refer to a chemical material or compound that inhibit HCV infection. As used herein, the terms “treatment,” “treating,” and the like, refer to obtaining a desired pharmacologic and/or physiologic effect. The effect may be prophylactic in terms of completely or partially preventing a symptom thereof and/or may be therapeutic in terms of a partial or complete cure for an adverse affect attributable to the condition. “Treatment,” as used herein, covers any treatment of an injury in a mammal, particularly in a human, and includes: (a) preventing HCV infection, arresting any complications, and minimizing its effects; (b) relieving the symptoms; (c) preventing the disease from occurring in a subject which may be predisposed to the disease but has not yet been diagnosed as having it; (d) inhibiting the disease, i.e., arresting its development; and (e) relieving the disease, i.e., causing regression of the disease.

As used herein, the term “individual,” “host,” “subject,” and “patient” are used interchangeably herein, and refer to a mammal, including, but not limited to, murines, simians, humans, mammalian farm animals, mammalian sport animals, and mammalian pets.

As used herein, the term “effective amount” or “therapeutically effective amount” means a dosage sufficient to provide treatment of the disease state being treated or to otherwise provide a desired pharmacologic and/or physiologic effect.

These and many other variations and embodiments of the invention will be apparent to one of skill in the art upon a review of the appended description and examples.

### EXAMPLES

### Example 1

### HCV E2 is a Novel Kinase and Interacts with AP50

The HCV E2 glycoprotein is a novel kinase that initiates signal transduction mechanisms modulating the following pathways: 1) Clathrin-mediated endocytosis, through a site-specific phosphorylation of the clathrin adaptor protein-50 (AP50), a key regulator of clathrin-mediated receptor endocytosis; and 2) Hepatocyte proliferation and liver carcinogenesis through the activation of PI3 Kinase and Akt. See WO 2007/101103, the entire application is incorporated by reference herewith.

In transfection studies and in vitro kinase assays, it is suggested that E2 is a novel member of the actin-regulating kinase family (Ark/Prk kinases) that associates physically with, and phosphorylates AP50 on its phospho-acceptor Thr156, a key step for clathrin-mediated endocytosis (25,50,73). Also, E2 is shown to be associated with AP50 in livers from HCV-infected patients, and that AP50 is phosphorylated on Thr156 to a much greater extent in these livers. It was also found that E2, in the absence of extracellular growth factors, increases PIP₂, PI3K, PDK1 and Akt, as well as their activities. This signaling cascade promotes proliferation. Moreover, HCV E2 markedly stimulates hepatocyte DNA replication to an even greater extent than classic tumor promoters TGFα and EGF.

The mechanisms by which HCV E2 associates with and phosphorylates AP50 thereby modulating clathrin-mediated endocytosis, and how E2 induces the PI3K pathway and increases hepatocyte proliferation, thereby facilitating liver carcinogenesis were studied. The physiological relevance of the interaction between HCV E2 and AP50 and the phosphorylation of AP50 in a unique HCV infection system of primary human hepatocytes were also studied. The protein motifs of E2 which are indispensable for association with and phosphorylation of AP50 was analyzed in the HCV Huh-7 infection system. The association and co-localization of E2 and AP50 by co-immunoprecipitation and immuno-staining of E2 and AP50 were also studied in a novel HCV infected primary hepatocyte system. The phosphorylation of AP50 by E2 with blocking antibodies and dominant negative peptides was further studied, and the protein domains of E2 were determined to be required for its association with AP50, and/or to be critical for its kinase activity by mutational analysis of E2 in the HCV Huh-7 infection system.

The effects of HCV E2 on proliferation in HCV infected primary human hepatocyte cultures and in the HCV Huh-7 infection system were studied. The effect of E2 on hepatocyte proliferation and DNA replication was also investigated in the novel HCV infected primary hepatocyte system, and through mutational analysis, sequences of E2 that are required for the induction of DNA replication were also determined in the Huh-7 HCV infection system. The effect of E2 on the PI3K/Akt pathway was also determined in the primary human hepatocyte HCV infection system, and the protein domains of E2 that are required for its activation were also determined in the Huh-7 HCV infection system.

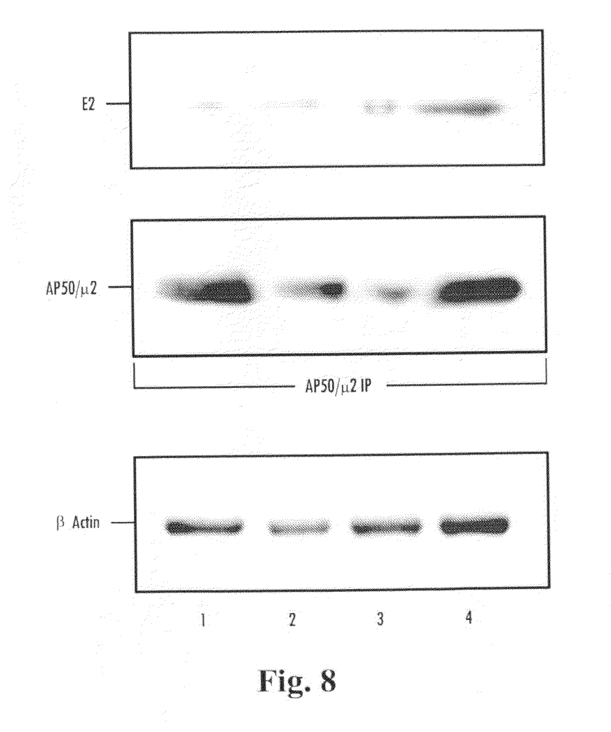

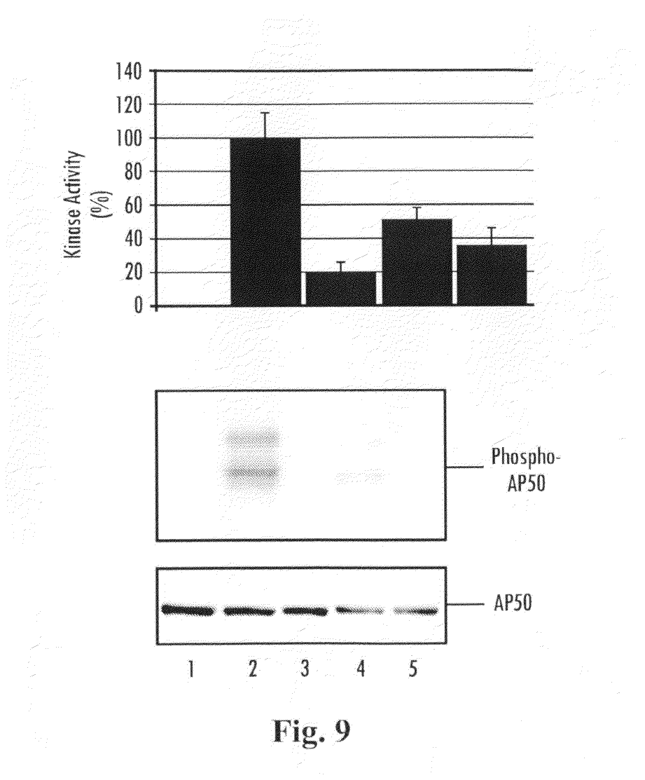

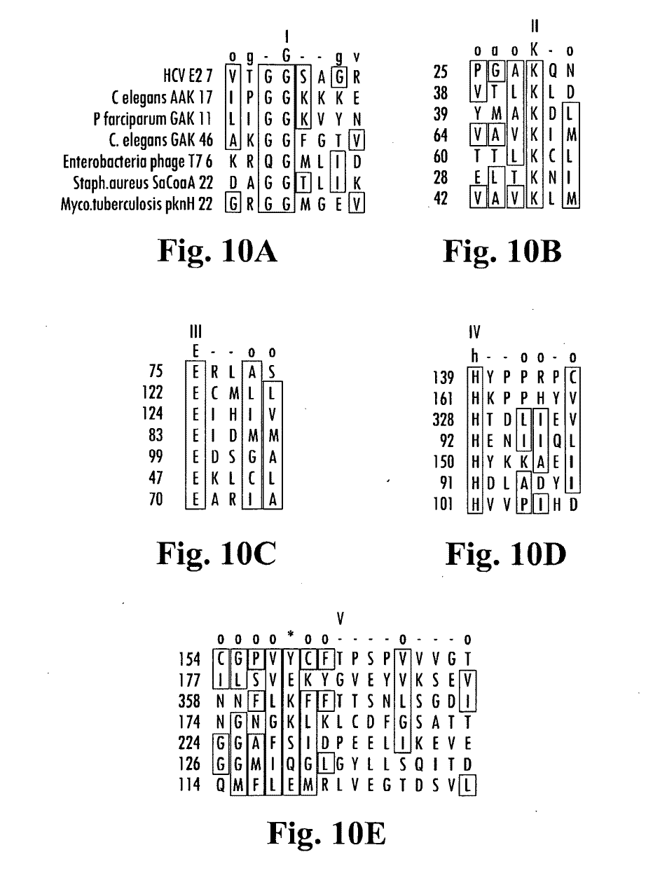

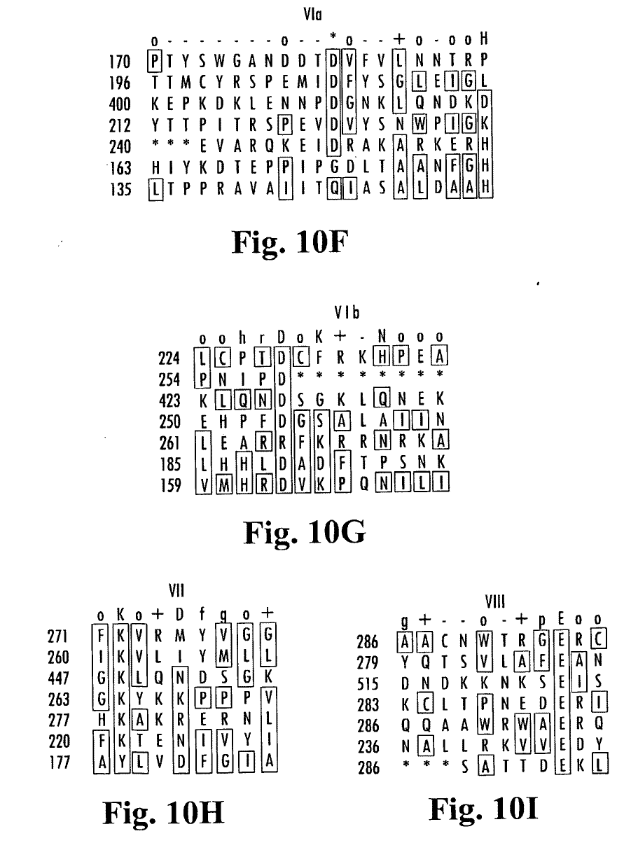

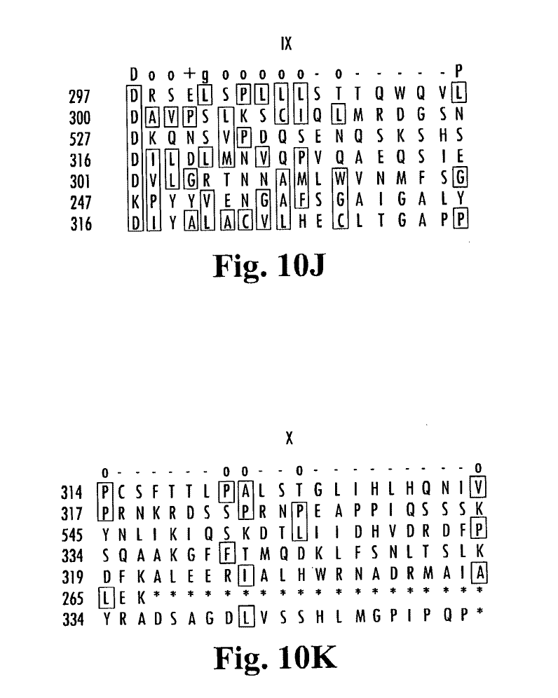

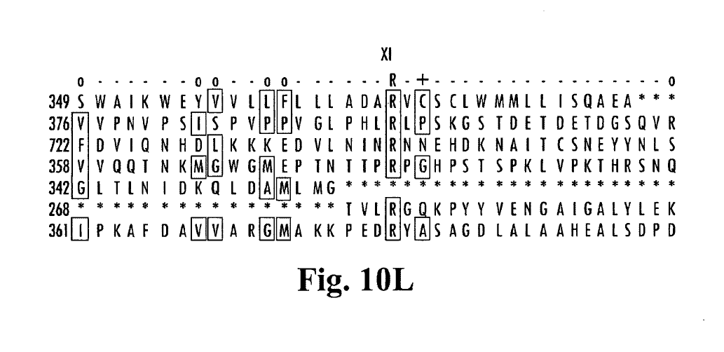

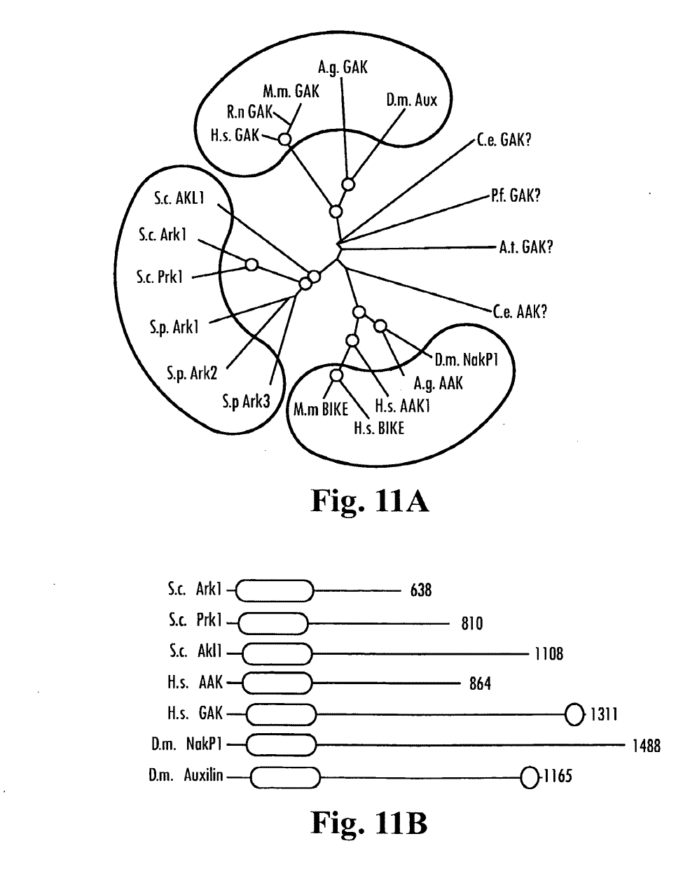

HCV E2 was also found to be a kinase (67), (15). In classical transfection and co-immunopurification experiments, it was shown that E2 is able to associate with AP50 (FIG. 8). In vitro kinase assays, it was also shown that HCV E2 is able to phosphorylate a (L(I)XXQXTG) (SEQ ID NO:41) consensus phospho-acceptor, present in AP50 (FIG. 9). The HCV E2 amino acid sequence was examined: it contains not only the II kinase domain of CDK but also all 11 structural domains that characterize all kinases (27) (26) (FIG. 10). Because E2 is a kinase that is able to phosphorylate the AP50 phospho-acceptor consensus site and it has some functional homology to GAK, it is a novel member of the Ark/Prk family of kinases (FIG. 11). The phytogenetic tree and the diagram of Ark/Prk1 kinase domains are depicted in FIG. 11.

The Ark/Prk family of kinases is known for their ability to phosphorylate proteins involved in clathrin mediated endocytosis. Through this phosphorylation, these kinases are able to control clathrin mediated endocytosis. The loose homology within their kinase domains is the only structural similarity that they share and as expected, this homology decreases as the gap between species widens. For example, there is a greater similarity among eukaryotes than among the very diverse prokaryotes. These homologies appear to be present, if more divergent in E2, the novel viral Ark/Prk family member. However, HCV E2 does contain the eleven domain structure that characterizes the eukaryotic protein kinases (27) (26) (37) (FIG. 10A-L).

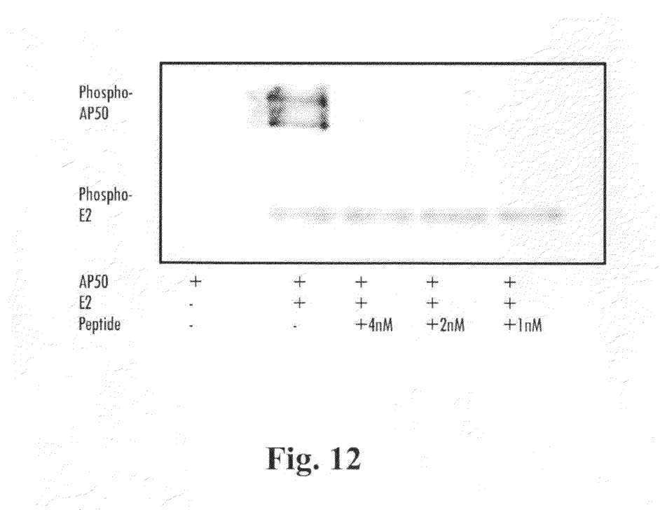

HCV E2 satisfies the major criteria established by Hanks and Hunter (26) for all protein kinases: 1) it contains the prerequisite structural elements, (FIG. 10, A-L); 2) it is associated with the protein(s) that it phosphorylates (FIGS. 8, 13 and 14); and 3) it kinases these protein(s) at a specific consensus phospho-acceptor motif, which can be assayed in an in vitro kinase system (FIGS. 9 and 12). Additional evidence that E2 is a kinase was also obtained. In some studies, E2 kinase activity was blocked with a dominant negative peptide of the E2 consensus phosphorylation site (FIG. 12).

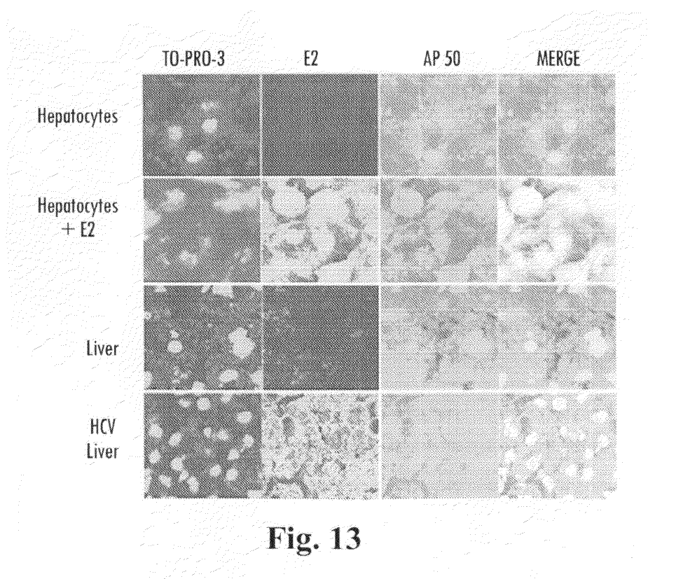

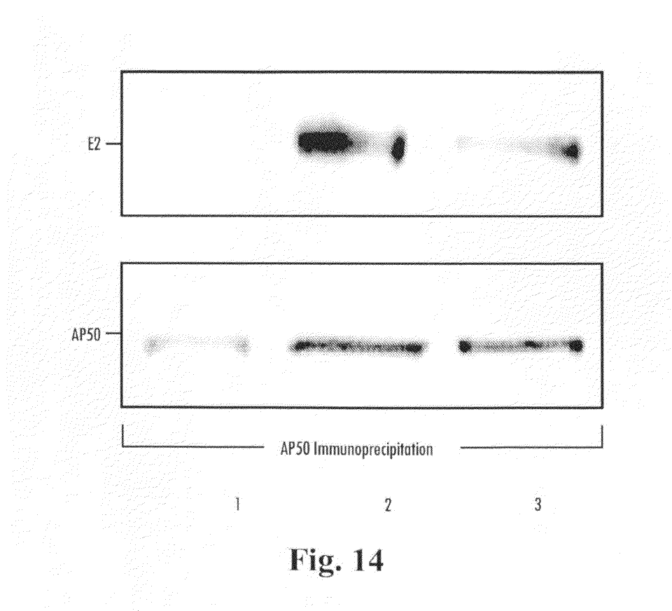

To assess the physiological relevance of the findings, whether HCV E2 is also associated with AP50 was also studied in the liver of HCV-infected patients. HCV E2 associated with AP50 in HCV-infected livers, as determined by co-localization using laser scanning confocal microscopy (FIG. 13, merge), and by co-immunoprecipitation assays (FIG. 14, lanes 2 and 3).

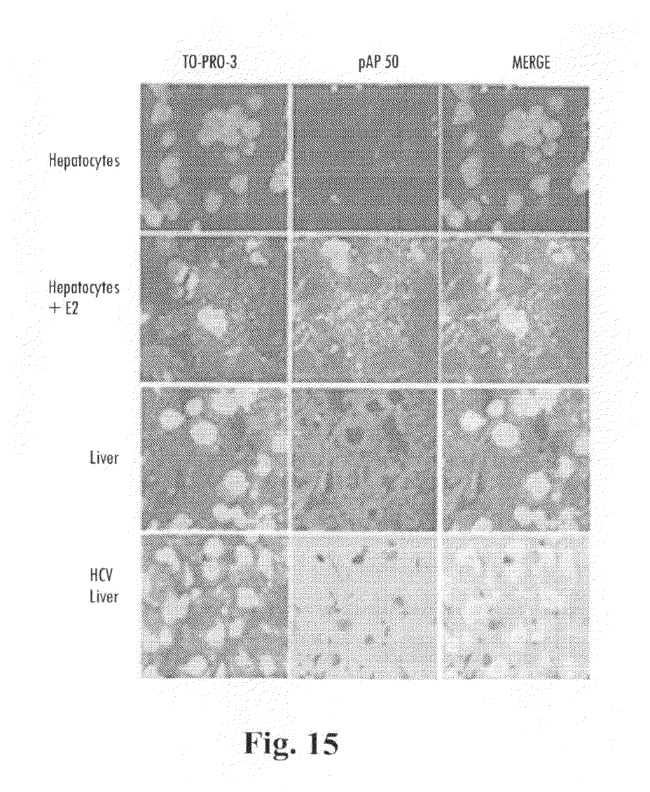

It was also found that HCV E2 is able to associate with AP50 in both transfected hepatocytes and in the livers of HCV infected patients (FIG. 13). In addition, AP50 is phosphorylated on threonine 156 in transfected primary mouse hepatocytes expressing E2 and in liver biopsies from patients infected with HCV (FIG. 15). A non-phosphorylatable mutant AP50 peptide of the phospho-acceptor site that binds to E2 with high affinity is able to block the phosphorylation of AP50 by recombinant E2 in vitro (FIG. 11). This is further proof that E2 is a kinase and that AP50 is one of its substrates.

The co-localization and association of E2 and AP50 were shown in vitro (FIG. 9), in transfected mouse hepatocytes (FIGS. 8 and 13), and in the livers of HCV infected patients (FIGS. 13 and 14). However, it is important to investigate this association in the context of the entire HCV instead of the E2 protein alone. Therefore, the interaction between E2 and AP50 is further characterized in HCV infected primary hepatocyte cultures and Huh-7 HCV infection system as follows.

The association of E2 and AP50 is studied in HCV infected primary human hepatocytes by co-immunopurification and immuno-staining with specific antibodies. In order to document that this association is direct, the association is blocked by Chariot E2 antibody transfection or microinjecting E2 antibodies before the HCV infection. These antibodies should block the intracellular association of E2 and AP50 without affecting HCV infection. It is not possible to selectively silence the E2 by siRNA since it exists as part of the HCV RNA.

### Example 2

### Dominant Negative Peptide of AP50 and E2 Mutants on HCV E2 Activity

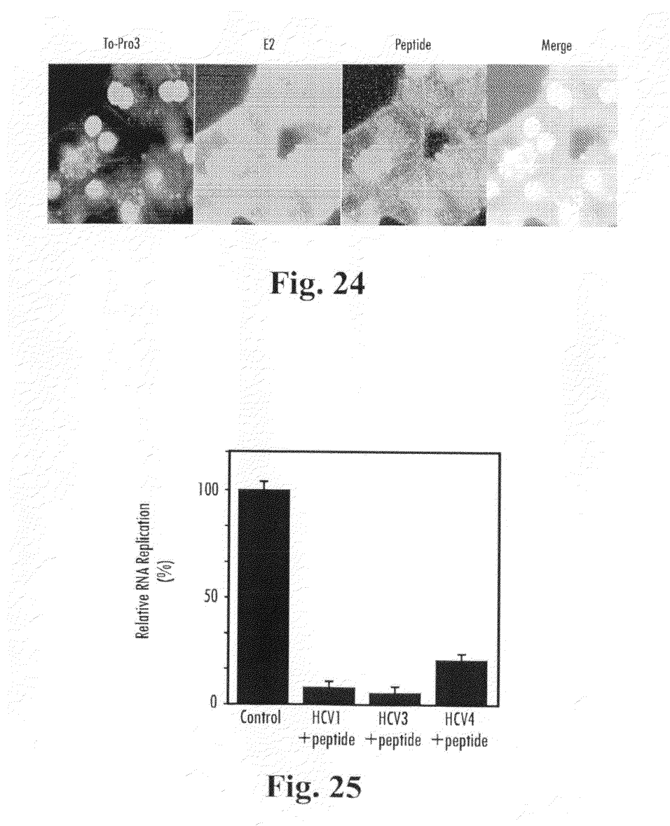

The dominant negative peptide of the AP50 phospho-acceptor (custom peptide synthesized by Celtek Peptides) is used that blocks the in vitro kinase activity of E2 (FIG. 12) to inhibit the intracellular association of E2 and AP50 in the HCV infected primary hepatocytes. Studies in HCV infected primary human hepatocytes have shown that this peptide is cell permeable (as it has an HIV tat leading sequence) and associates with E2 (FIG. 24). FIG. 24 shows that the peptide is in the perinuclear region of the ER, co-localized with HCV E2.

The sequences of E2 that are required for its association with AP50 and/or are important for its kinase activity were determined by mutational analysis. These additional mutations were assessed in the in vitro kinase assay and in the primary hepatocyte transfection studies. The E2 point mutations that disrupt the phosphorylation of AP50 and/or the induction of proliferation were assessed in the Huh-7 HCV infection system.

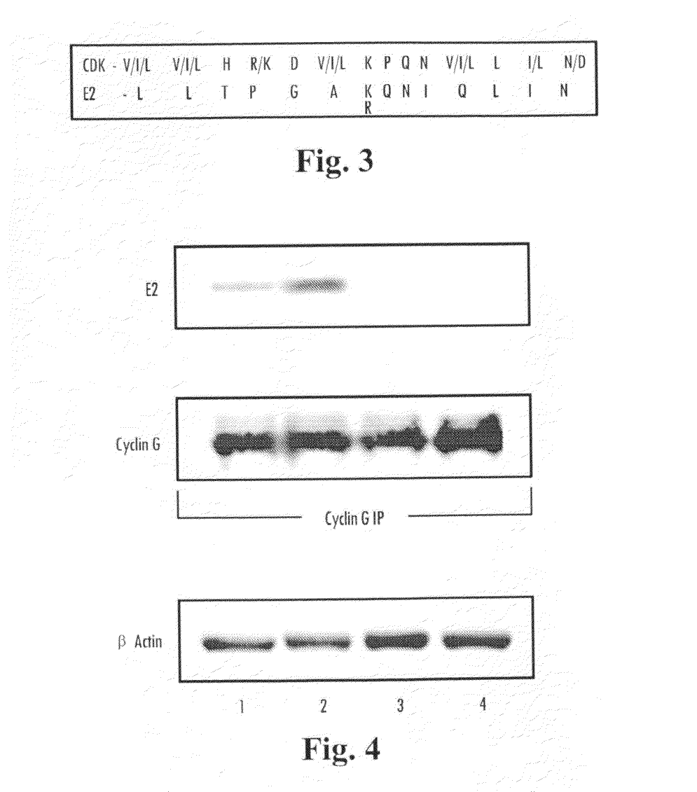

Three important mutants, K25R, Y228E and Y228F, were expressed and shown in FIGS. 3-5, 8-10, and 18 using the Roche RNA Translation System, which allows an efficient production of proteins. When introduced into hepatocytes, these proteins undergo the appropriate post-translational modification and localize to the expected organelles. In the Huh-7 HCV infection system, cells were transfected with mutated HCV RNA resulting in the production of infectious HCV pseudo particles (75) (71).

All of the additional nine E2 mutants replacing 5 leucines, 2 isoleucines, 1 glutamic and 1 aspartatic with alanines, as well as deleted E2 constructs were produced as previously described (8; 9) (12). These mutations include; K25R and D275A, putative kinase domains II and IX conserved amino acids respectively, Y228E/F and L282A, a putative cargo domain and a putative di-leucine based motif that are presumed to facilitate the association of E2 with AP50, L197A, E272A, L283A, L292A, I313A, I331A, and L342A, all amino acid motifs contained in E2 that are also in the kinase domain of GAK.

The ability of purified mutant recombinant E2 proteins to associate with purified recombinant AP50 is quantified in co-immunoprecipitation assays (8; 11; 17). The association of E2 with AP50 was also studied in primary human hepatocytes, after transfection of C-terminal or N-terminal deletions, as well as appropriate mutations, using transfection reagents as described previously (8) (33; 34). To ensure accurate protein identification, E2 and AP50 specific antibodies were utilized. In addition, the biological relevance of these protein/protein interactions on AP50 Thr¹⁵⁶ phosphorylation, and whether phosphorylation of AP50 Thr¹⁵⁶ alters its affinity to E2 were analyzed.

In brief, the E2:AP50 interactions were studied as follows (see also Methods): a) Interaction of recombinant wild type, deleted and mutated proteins in cell-free systems, and after DNA or protein transfection into primary hepatocytes. Relevant mutations discovered in this assay were analyzed in the Huh-7 HCV infection system; b) Co-immunoprecipitation with specific antibodies as described (8; 11). An antibody that recognizes the phosphorylated Thr¹⁵⁶ phosphoacceptor but not the unphosphorylated domain of AP50 was obtained (66; 73). Direct and reversed ‘pull-down’ studies were performed as described previously (8; 9). This documents changes in direct or indirect association between E2 and AP50, and in the phosphorylation state of AP50; c) Confocal scanning laser microscopy with specific antibodies as described by the PI (8; 10; 12). This evaluates changes in co-localization, cellular location, and effects throughout the cell population induced by the mutations; and d) phosphorylation of AP50 Thr¹⁵⁶ by E2 is studied in cell-free systems as well as in HCV infected primary hepatocytes and the Huh-7 HCV infection system. Phosphorylation of AP50 Thr¹⁵⁶ is determined with ³²P-γATP, with specific antibodies against the phosphorylated domain (66; 73), and by Mass Spectroscopy.

It was found that HCV E2, after transfection into mouse hepatocytes, associates with AP50 (FIGS. 8 and 13). Also recombinant E2 is able to associate with an immuno-purified AP50, and phosphorylates AP50 on Thr¹⁵⁶ (FIG. 9, lane 2, and FIG. 12, lane 2), the same residue phosphorylated by GAK. Like most kinases, E2 can also self-activate through autophosphorylation (data not shown).

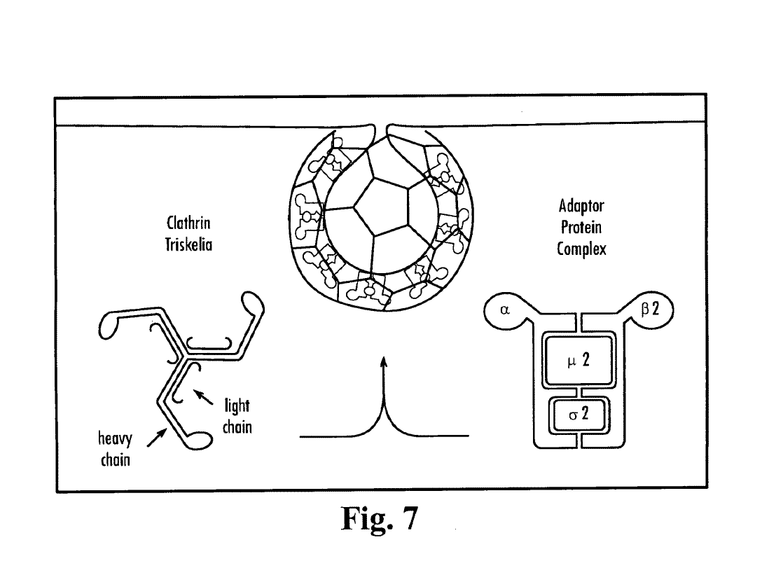

The role of the eight conserved domains between GAK and E2 was assessed by deletion and mutational analysis (FIGS. 3-5, 8-10, and 18). In addition, the tyrosine to glutamic (Y228E) and to phenylalanine (Y228F) mutation in one of the AP50 binding motifs (cargo domain), and a lysine to arginine mutation (K25R) in the HCV E2 catalytic loop were analyzed. Some of these mutants were markedly impaired endocytosis in the Huh-7 HCV infection system due to their inability to phosphorylate AP50 (FIG. 9) and connect the cell membrane signals to the clathrin β subunit (FIG. 7). In cell-free systems, these E2 mutants are either not to bind, or to bind but not to phosphorylate AP50. The mutations that have been found to be relevant in the in vitro recombinant protein assays and transfection studies of primary mouse hepatocytes are reproduced in the HCV genome used to infect the Huh-7 cells and studied in this system.

It was also found that mutations of lysine in domain II of the kinase catalytic loop (FIG. 9, lane 3) and the tyrosine in the AP50 binding motif/cargo domain (FIG. 9, lanes 4 and 5) decrease the phosphorylation of AP50 by E2. Therefore, it is provided that the E2 protein is a novel kinase that phosphorylates AP50 on Thr¹⁵⁶; a kinase catalytic loop mutant of E2 (K25R) would not phosphorylate AP50; a cargo domain mutant of E2 (Y228E/F) would not phosphorylate AP50; and it cannot be predicted whether other proposed eight E2 mutations of the conserved amino acids between GAK and E2 will associate with or phosphorylate APR50 .

Therefore, the effects of the E2 recombinant wild type and mutant proteins on AP50 Thr¹⁵⁶ phosphorylation with ³²P-γATP were studied in cell-free assays, as well as in primary mouse hepatocyte cultures with antibodies specific against the phosphorylated AP50. The association between E2 and AP50 was also studied by scanning confocal microscopy as well as by immunopurification and immunoblots, and the association of E2 and AP50 and the phosphorylation of AP50 in an unique HCV infection model using primary human hepatocytes and serum-derived virus. This documents the association and phosphorylation in the context of the entire HCV, rather than the E2 alone. Moreover, the relevant mutations described above reproduced in the HCV genome was used by the Huh-7 cells and investigated in this model to prove by point mutation analysis which E2 protein motifs are indispensable for E2/AP50 association and AP50 phosphorylation.

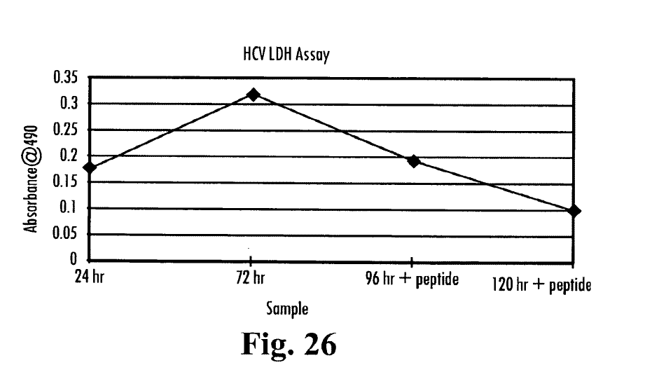

It was found that the dominant negative AP50 peptide inhibits HCV infection without inducing hepatocyte toxicity in the primary hepatocyte infection system. The efficacy of the dominant negative AP50 peptide in the inhibition of HCV infection in the primary human hepatocytes and in the Huh-7 infection models were also investigated (FIGS. 25 and 26).

### Example 3

### HCV E2 on PIP₂-PI3K/AKT Signaling and DNA Replication and Proliferation

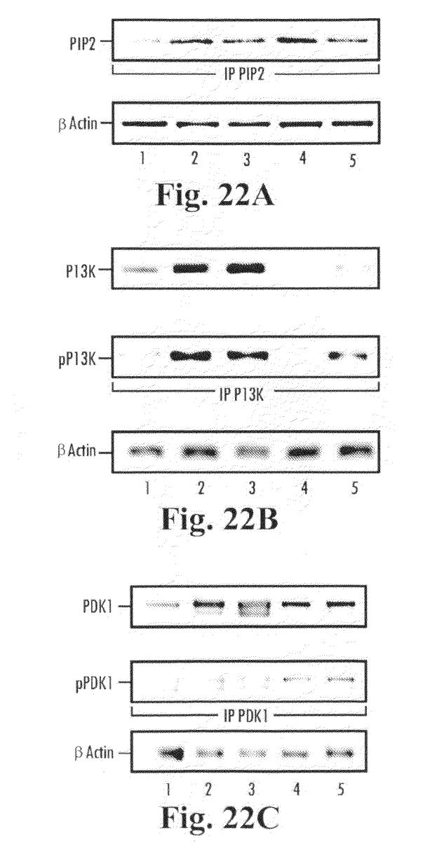

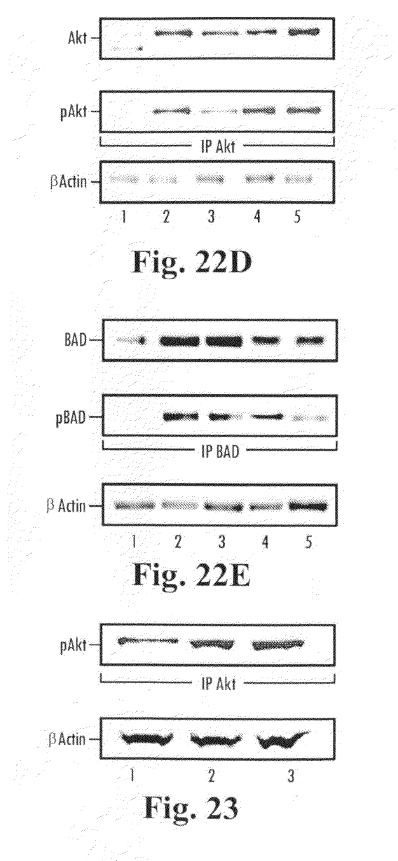

Phosphotidylinositol 4,5-biphoshate (PIP₂) is required for clathrin-mediated endocytosis (51) (72). PIP₂ is a phosholipid making up 1% of the cytoplasmic leaflet of the plasma membrane (46). The AP2 complex is recruited exclusively to PIP₂ anchored in the plasma membrane where AP2, through its AP50/μ2 subunit, when phosphorylated, binds to the cargo domains of receptors and incorporates them into the clathrin-coated endocytic vesicles. Honing and co-workers (31) have shown that AP2 binding to the cargo domains of receptors and acidic di-leucine clathrin motifs is contingent upon recognition of PIP₂. AP2 binds PIP₂ through its' α and μ2 subunits (60). Therefore, the role of HCV E2 on PIP₂ is also evaluated. It was found that HCV E2 transfected into mouse hepatocytes causes an increase in PIP₂ (FIG. 22A), which could contribute to the increased endocytosis of these cells (FIG. 20).

Activation of Akt/PKB has been strongly implicated in the initiation, progression, and prognosis of HCC. The PI3K/Akt/mTor pathway is responsible for the initiation and maintenance of uncontrolled cellular proliferation which is necessary for liver carcinogenesis (58). Akt is also a risk Factor for early recurrence and poor prognosis of HCC (49).

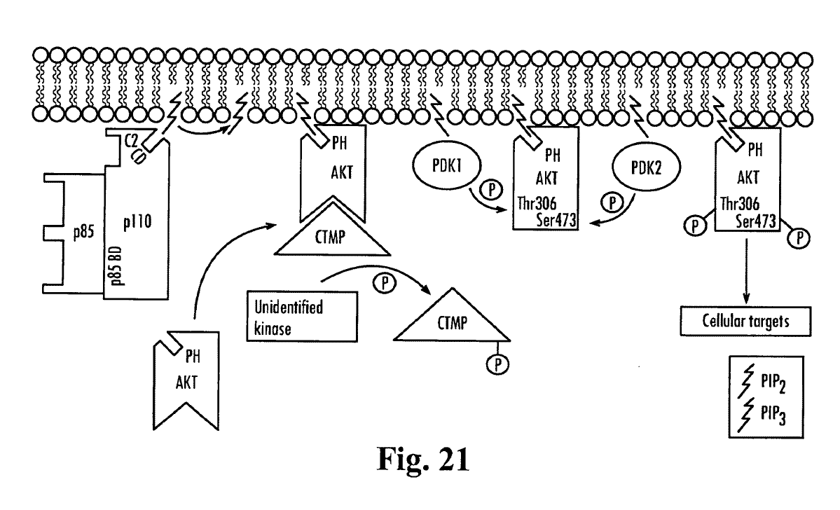

As depicted in FIG. 21, phosphoinositol 3 kinases (PI3K), principally a p110 catalytic subunit, becomes activated, usually through growth factor stimulation and converts PIP₂ to phosphoinositol-3,4,5-triphopshate (PIP₃). Signaling proteins with membrane binding pleckstrin-homology domains (PH), Akt and phosphoinositol dependent kinase 1 (PDK1) are recruited to activated PI3K, and activated PDK1 is able to activate Akt through phosphorylation (FIG. 21). Activated Akt phosphorylates a multitude of proteins that affect cell growth, cell cycle entry, and cell survival. Akt phosphorylates BAD, preventing its association with Bcl-2 and Bcl-XL, blocking apoptosis. PDK1 phosphorylates and activates other protein kinases, including p70 S6-kinase which activates the translation of cell growth genes (14) (70).

Therefore, the effect of HCV E2 on PI3K/AKT signaling is provided in the present invention. It was found that E2 not only increases PIP₂ (FIG. 22A), but also PI3K (FIG. 22B), PDK1, (FIG. 22C) and Akt (FIG. 22D), and their activities in the absence of extra-cellular growth factors. In addition, BAD is phosphorylated in cells given E2 (FIG. 22E). In brief, HCV E2 is not only a potent inducer of cell proliferation (FIG. 18), but also blocks the apoptosis cascade (FIG. 22E) through the activation of the PI3K/Akt signal transduction pathway. Of physiological relevance to these studies, it was found that the activation of Akt, a central kinase in the PI3K pathway, is increased in the livers of HCV infected patients (FIG. 23).

It was found that HCV E2, after transfection into primary mouse hepatocytes, increases the expression and activity of PI3K, PDK1, and Akt (FIGS. 22, B, C and D). Also E2 is able to increase the expression of PIP₂ (FIG. 22, A). The relevance of these studies is clear from the presence of active Akt in HCV-infected livers (FIG. 23). However, it is important to investigate this induction of the PI3K pathway in the context of an entire HCV instead of the E2 protein alone.

Therefore, the induction of the PI3K pathway was further characterized in HCV infected primary hepatocyte cultures and Huh-7 HCV infection system as follows: a) in order to document that this effect is direct, the effect was blocked by Chariot E2 antibody transfection or microinjecting E2 antibodies before infection. These antibodies should block the induction of the PI3K pathway without affecting HCV infection; b) the dominant negative peptide of the AP50 phospho-acceptor that blocks the in vitro kinase activity of E2 (FIG. 12) was used to assess whether E2 kinase activity is required to induce PI3K activity in the HCV infected primary hepatocytes. Studies in HCV infected primary human hepatocytes have shown that this peptide is cell permeable and associates with E2 (FIG. 24); and c) the sequences of E2 that are required for the induction of the PI3K pathway were determined by mutational analysis. These E2 point mutations are assessed in the Huh-7 HCV infection system.

The ability of E2 to induce the expression and activity of the PI3K pathway and the expression of PIP₂ were investigated using immuno-purification assays in HCV infected primary human hepatocytes and in wild type, mutated, and deleted E2 in the Huh-7 infection system. Expression and activity of the kinases in the PI3K pathway were evaluated using specific antibodies (see also Methods). The cellular location of these active kinases were analyzed by confocal microscopy using specific organelle markers (Plasma membrane-Integrin α2, GRP78-ER, EEA1-endosomes, Bcl-2-mitochondria, GM130-golgi, Nucleoporin62-nucleus, Caveolin1-caveolae, and Lamp1-lysosomes).

In brief, the effects of E2 upon the PI3K pathway were studied as follows (see also Methods): a) IP₂ was immuno-purified using specific antibodies from HCV infected primary human hepatocytes and from Huh-7 cells infected with HCV wild type, mutant, or deleted E2 and its expression was evaluated by immuno-blot using specific antibodies; b) I3K, PDK1, and Akt were immuno-purified using specific antibodies in HCV infected primary human hepatocytes, and from Huh-7 cells infected with HCV wild type, mutant, or deleted E2 and their expression and activity was evaluated by immuno-blot using specific antibodies; c) downstream kinases and targets of the PI3K pathway, such as BAD and GSK3β were immuno-purified using specific antibodies from HCV infected primary human hepatocytes and from Huh-7 cells infected with HCV wild type, mutant, or deleted E2 and their expression and activity were evaluated by immuno-blot using specific antibodies; and d) the above kinases and downstream targets of this pathway were localized within the cell by confocal scanning laser microscopy in HCV infected primary human hepatocytes and from Huh-7 cells infected with HCV wild type, mutant, or deleted E2 as previously described (8; 10; 12).

It was found that the wild type HCV E2 stimulates the PI3K/Akt signaling cascade and DNA replication in HCV infected human hepatocytes. HCV E2 mutations of lysine in domain II of the kinase catalytic loop (K25R) and the tyrosine in the AP50 binding motif/cargo domain (Y228E/F) of E2 did not stimulate the PI3K/Akt signaling nor the cell proliferation in the Huh-7 infection system. The effects of HCV E2 wild type and mutants on PI3K/Akt signaling were also studied by confocal microscopy and immunoblotting for these kinases and their active, phosphorylated moieties. The effects of HCV E2 wild type and mutants on DNA replication were also studied by [³H]-thymidine incorporation into DNA as well as by analysis of the cell cycle by cell sorting. The mass spectroscopy was used to analyze kinase activation. Different amino acid substitutions were investigated if the original mutations induce toxicity that cannot be mechanistically explained. PCNA was replaced with other antibodies to markers of proliferation (MPP-2 and ki-67) if necessary, and Brdu was used as a label of S-phase if [³H] thymidine was found to be insensitive or toxic.

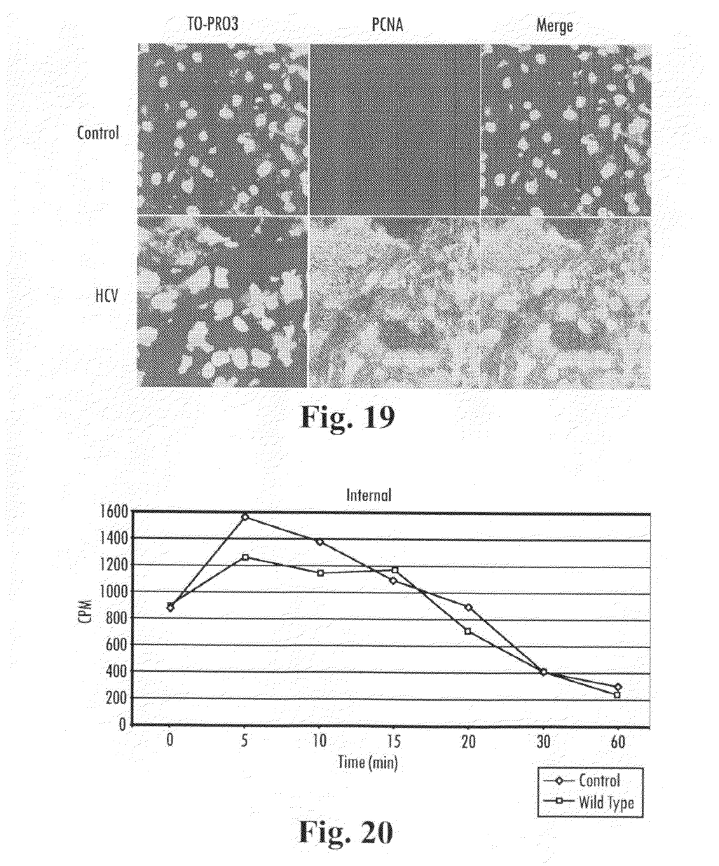

It was shown that E2 increases DNA replication in transfected mouse hepatocytes (FIG. 18), and that hepatocytes in the livers of HCV infected patients were in S-phase (FIG. 19). However, it is important to investigate whether hepatocytes proliferate in the context of the entire HCV instead of the E2 protein alone.

Therefore, DNA replication was further characterized in HCV infected primary hepatocyte cultures and Huh-7 HCV infection system as follows: a) DNA replication was studied in HCV infected primary human hepatocytes by [³H]-thymidine incorporation assays and immuno-staining for PCNA (proliferating cell nuclear antigen) with specific antibodies. In order to document that this effect is direct, the association was blocked by Chariot E2 antibody transfection or microinjecting E2 antibodies before infection. These antibodies should block the DNA replication and cell entry into S-phase without affecting HCV infection; b) the dominant negative peptide of the AP50 phospho-acceptor that blocks the in vitro kinase activity of E2 (FIG. 12) was used to inhibit the E2 induced DNA replication in the HCV infected primary hepatocytes. Studies in HCV infected primary human hepatocytes have shown that this peptide is cell permeable and associates with E2 (FIG. 24); and c) the sequences of E2 that are required for the induction of DNA replication and cell entry into S-phase were determined. These E2 point mutations were assessed in the Huh-7 HCV infection system.

Mutations of the protein motifs of E2 found to be relevant in primary hepatocyte transfection studies were reproduced in the HCV genome used in the Huh-7 cells and evaluated in this infection system. The effects of E2 upon proliferation and cell cycle were further characterized by cell sorting. E2 wild type or mutants were infected and sorted by flow cytometry according to DNA content and size, effectively quantifying the percentages of cells in Go, Gl, S, and M phases. This demonstrates the effects of E2 upon the cell cycle of a normal hepatocyte (see also Methods) and documents which E2 protein motifs are important for cell proliferation in the HCV infected Huh-7 system.

In brief, the effects of E2 upon DNA replication and proliferation were studied as follows: a) [³H]-thymidine incorporation assays of E2 in HCV infected primary human hepatocytes and mutations in the Huh-7 system; b) Confocal scanning laser microscopy with specific antibodies to E2 and PCNA as previously described (8; 10; 12); and c) Flow cytometry assays of E2 in HCV infected primary human hepatocytes and mutations in the Huh-7 system

### Example 4

### HCV E2 Motifs and Mutants on Autophosphorylation and Phosphorylation of AP50

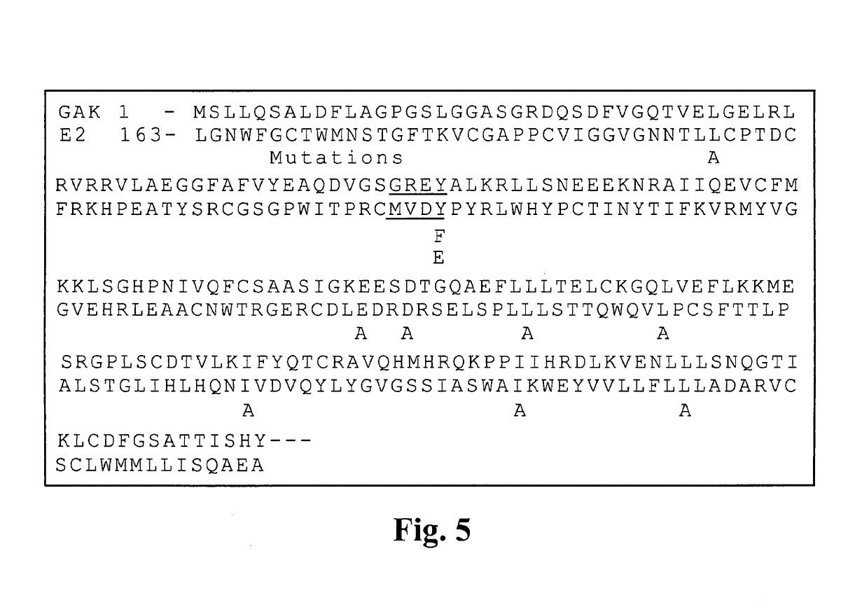

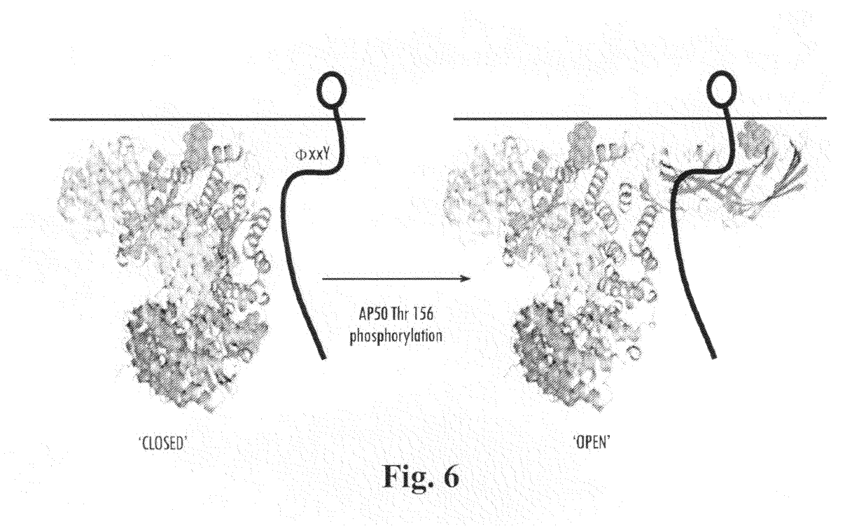

It was further found that HCV E2 has a putative cargo (ΦXXY) domain (FIGS. 5 and 6) and a leucine based motif ((D/E)XXXL(L/I)) (SEQ ID NO:35). These motifs are known to facilitate membrane receptor interaction with AP50 (50). They are conserved in all of the HCV genotypes (data not shown). E2 is the first protein identified to contain a functional receptor cargo domain and a di-leucine based motif that is not a membrane-associated receptor. Thus, it is postulated that E2 may be acting as a surrogate cellular receptor for HCV internalization.

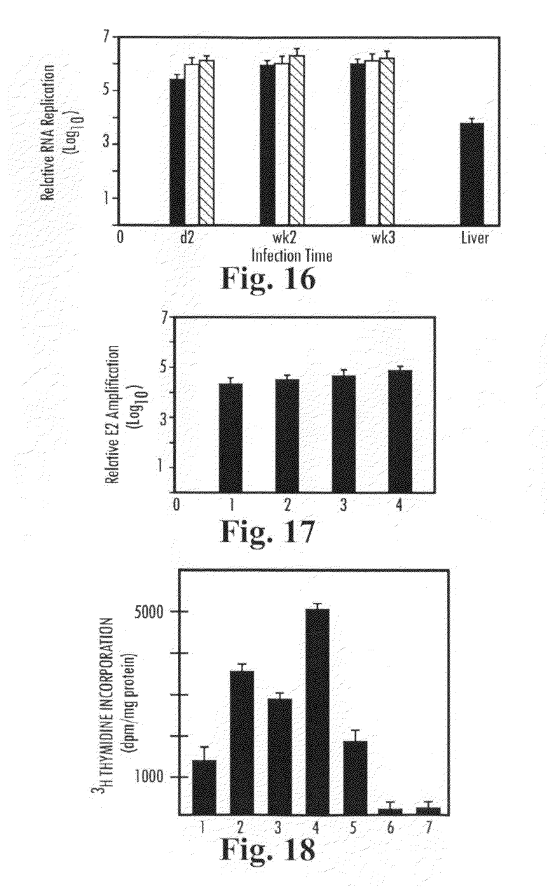

A mutation of either the putative (ΦxxY) cargo domain (Y228E/F) (FIG. 5) or the ((D/E)XXXL(L/1)) (SEQ ID NO:35) leucine-based domain (L282F) decreases the autophosphorylation of E2. Therefore these mutations also decrease the kinase activity of E2 and the corresponding phosphorylation of AP50 on threonine 156 (FIG. 9). The physiological relevance of these motifs was investigated in an unique HCV infection system of primary human hepatocytes (FIGS. 16 and 17), and mutation of these domains in the Huh-7 HCV infection system provided valuable insights into the importance of these motifs in the HCV lifecycle. These experiments using Y228E/F (putative cargo domain) and L282A (putative di-leucine AP50 binding domain) would evaluate the importance of these motifs in the E2 association with and phosphorylation of AP50 as well as in the induction of the PI3K pathway. The conserved kinase motifs (K25R and D275A) and the amino acid domains found in both E2 and GAK (L197A, E274A, L283A, L292A, I313A, I331A, and L342A) would also be mutated to assess their roles in E2/AP50 association, the phosphorylation of AP50, and cell proliferation.

HCV E2 motifs provided in the present invention include kinase domain K25R, cargo domain Y228E/F, and di-leucine based motif L282F mutations, among other motifs that are homologous to either GAK or are conserved kinase domains. The Huh-7 derived replicon systems are valuable to study the molecular mechanisms mediating HCV infection. A mutational analysis was used in this system to determine the importance of the identified E2 motifs. The mutations were also used to clarify the role that these motifs have in E2/AP50 association, AP50 phosphorylation by E2, and the induction of the PI3K pathway by E2.

It was noted that several of the mutants were unable to induce proliferation in transfection studies, notably K25R in the kinase catalytic loop, and Y228E/F in the cargo domain (FIG. 18), suggesting that these motifs in E2 are individually necessary for the increase in cellular proliferation. These data also imply that an induction of endocytosis, in the absence of growth factors, can stimulate abnormal proliferation and that a blockade of E2 with a dominant negative AP50 peptide would inhibit HCV infection (See WO 2007/101103, the entire application is incorporated by reference herewith).

These recently characterized HCV E2 mechanisms have yet to be explored in an HCV-infected normal primary human hepatocyte model system. This will be a valuable model to study these intriguing HCV E2 mechanisms, and possibly others, in the presence of the entire, naturally occurring HCV viral particle with a complete life cycle, obtained directly from patients. Mutational analysis in the Huh-7 HCV infection system is necessary in order to investigate the roles of the individual motifs of E2 and their importance in HCV infection. In addition, the discovery and mechanistic studies of a novel viral kinase has extensive implications in the fields of HCV, general virology, clathrin-mediated endocytosis, and signal transduction.

A unique HCV infection system was developed utilizing serum derived virus and normal primary human hepatocytes, this system will be used to explore the physiological relevance of HCV E2 association and phosphorylation of AP50. A detailed description of this unique HCV infection system can be found in WO 2007/101103, the entire application is incorporated by reference herewith.

A primary hepatocyte cell culture susceptible to the induction of cell proliferation was used. The HCV E2 protein was determined to induce hepatocyte proliferation in normal primary hepatocytes (FIG. 18). Expression of HCV E2 in primary mouse hepatocytes was sufficient to induce cell entry into S-phase, as determined by the incorporation of [³H]-thymidine into DNA (FIG. 18, lane 4). The effects of HCV E2 on hepatocyte proliferation exceeded those induced by the tumor-promoters TGFα and EGF (36; 62) in the same experiments (FIG. 18, lanes 2 and 3). Further, these effects of HCV E2 on hepatocyte proliferation were confirmed by analyzing the cell cycle; expression of E2 stimulated cell entry into S-phase (data not shown). The relevance of these studies was supported by the findings in liver biopsies from patients infected with HCV. Hepatocytes in HCV infected livers were found to be in S-phase also, as indicated by nuclear staining of PCNA, (8). (FIG. 19, bottom middle panel). These findings suggest that HCV E2 plays an important role in the stimulation of liver tumorigenesis by HCV. The effects of serum derived HCV in the unique primary human hepatocyte infection model were also investigated (FIGS. 16 and 17). Mutations of the above described motifs could be used in a serum-starved model of the Huh-7 HCV infection system.

### Example 5

### HCV E2 and Transferrin Internalization

It is known that the phosphorylation of AP50 on threonine 156 is important for transferrin receptor endocytosis (48). Extracellular iron circulates in plasma bound to transferrin (Tf), and it is internalized in the hepatocytes through transferring receptor-2 (TfR2), clathrin-coated pit regulated endocytosis (30). It was found that the HCV E2 protein also increases the internalization of Tf in primary hepatocytes. In primary hepatocytes transfected with the E2 expressing cDNA, the internalization of [¹²⁵I]-Tf, is faster and significantly greater than in control hepatocytes without E2 expression (FIG. 20). Because the amount of total surface-bound Tf remained unchanged (data not shown), this increased Tf uptake reflects an induction of early endocytosis.

Eukaryotic cells require iron for growth and survival. Hepatocytes are important in systemic iron homeostasis as the liver is a major storage source of iron. Mutations in the human Tf R2 gene result in Hemochromatosis, characterized by iron overload in the liver leading to cirrhosis and cancer (13). The ability of the E2 protein to regulate the internalization of Tf, and with it the entire protein-iron complex, ensures sufficient iron for hepatocyte proliferation and survival and may impart some beneficial effects to the invading HCV pathogen as well.

Indeed, patients with chronic HCV infection have been shown to accumulate iron (22; 38). Interestingly, transgenic mice expressing the HCV polyprotein, when achieving iron overload levels similar to those found in HCV-infected patients, develop mitochondrial injury and an increased risk of hepatocellular carcinomas (23). The levels of iron accumulation in HCV patients and those achieved in the transgenic studies were moderate. The amount of increased transferrin endocytosis that was found in the E2-transfected primary mouse hepatocytes could easily account for these moderate levels of iron overload that lead to increased risk of hepatocellular carcinomas and mitochondrial injury.

It is demonstrated that HCV E2 controls the clathrin-mediated endocytosis of transferrin, an archetype of CME, in transfected primary mouse hepatocytes. Studies in these HCV models could elucidate whether the E2 is able to control endocytosis in a physiological model of HCV infection. A specially designed AP50 peptide can be used to block E2 activity and investigate its individual contribution in this HCV infected primary hepatocyte model. Mutational analysis could be used in the Huh-7 HCV infection model.

### Example 6

### Phospho-Peptide Mapping of HCV E2 Kinase

Identification of HCV E2 as a kinase by typical in vitro kinase assays and structural domain analysis gives tremendous insight into E2's potential mechanisms. These data identify the phosphor-acceptor sites of E2 and place it in a kinase family with a defined role in endocytosis.

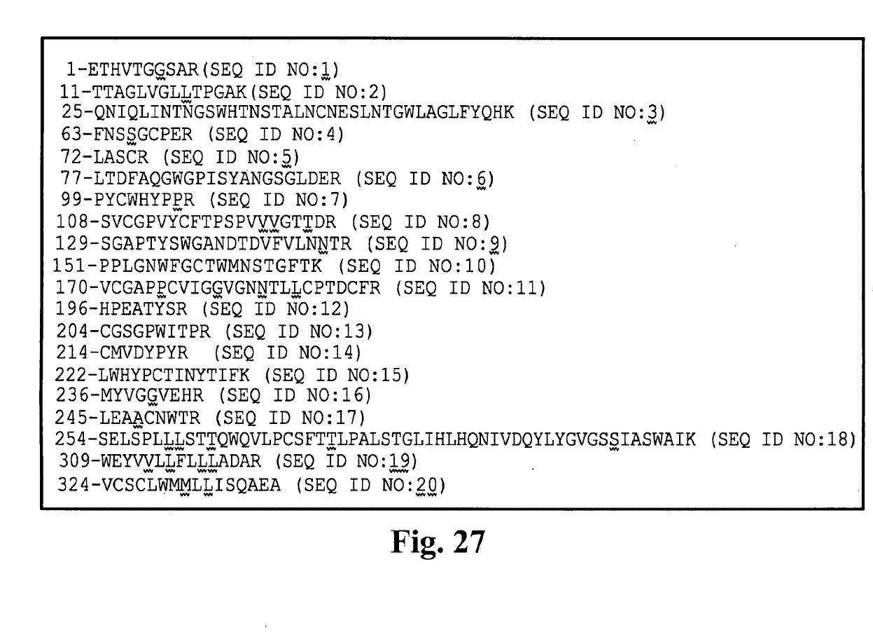

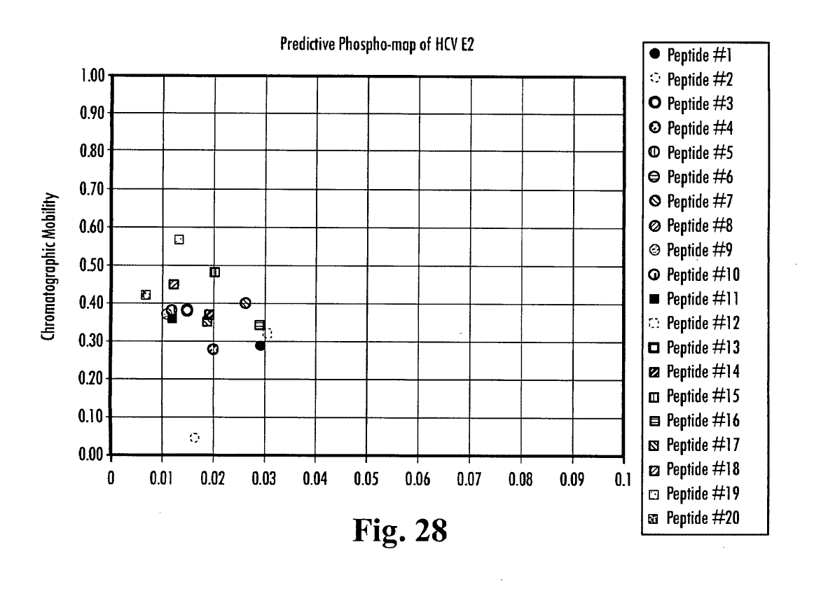

Moreover, a phospho-peptide mapping with [³²P]-γ-ATP of all of the putative phosphorylation sites of the E2 kinase was provided (FIGS. 27 and 28). This identifies potentially important phosphorylation sites of E2. Once the phosphorylation sites are established, their relevance to HCV infection can be explored. Mutations of these phosphorylation sites were made and evaluated in the Huh-7 infection system to test their impact on E2 function. Antibodies were produced and evaluated for their ability to block HCV E2 mediated cell entry and therefore HCV infection. These studies lead to a passive or active immunization for HCV infection.

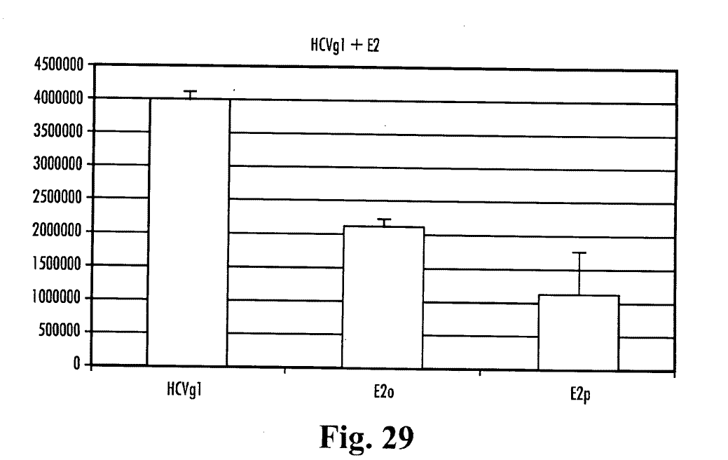

FIG. 29 shows antibody blockage of HCV infection in the primary human hepatocytes with genotype 1 patient serum. Antibodies were made to the tyrosine (Y) and surrounding motif of peptide 14 (214-CMVDYPYR) (SEQ ID NO:14). E2o antibody was made to an unphosphorylated motif and antibody E2p was made to a phosphorylated motif.

### Methods

### Human Primary Hepatocyte Cultures

Hepatocytes were obtained (from Tissue Transformation Technologies [Edison, N.J.]) from anonymous organ donors without liver disease that were not suitable for liver transplantation for technical but not medical reasons. These donors are negative For Hepatitis A, B and C, CMV, HIV, HTLV ½, and RPR-STS. Hepatocytes cultures with >5% apoptosis by annexin-V assays and/or increases >3-fold in ALT were discarded.

Hepatocytes were isolated from an encapsulated liver sample by a modified two-step perfusion technique introduced by Seglen (63). Briefly, the dissected lobe was placed into a custom-made perfusion apparatus and two to five hepatic vessels were cannulated with tubing attached to a multi-channel manifold. A liver fragment (150 to 500 g) was perfused initially (recirculation technique) with calcium-free HBSS supplemented with 0.5 mM EGTA for 20 to 30 min and then with 0.05% collagenase [Sigma] dissolved in L-15 medium (with calcium) at 37° C. until the tissue was fully digested. The digested liver was removed, immediately cooled with ice-cold L-15 medium and the cell suspension was strained through serial progressively smaller stainless steel sieves, with a final filtration through 100-micron and 60-micron nylon mesh. The filtered cell suspension was aliquoted into 250-ml tubes and centrifuged three times at 40 g for 3 min at 4° C. After the last centrifugation, the cells were re-suspended, in HypoThermosol-FRS [BioLife Solutions, Inc] combined in one tube and placed on ice.

Cells were centrifuged at 700 rpm for 5 min at 4° C., the supernatant was removed and the cells were washed with Hanks Wash Solution (53.6 mM KCl 0.4 g/l; 4.4 mM KH₂PO 0.06 g/l; 1.37 M NaCl 8 g/l; 3.4 mM Na₂HPO₄ 0.048 g/l; 20 μL CaCl₂ (2M)) three times. Cells were re-suspended in Hepatocyte Plating Media (500 mL DMEM high glucose; 20% FBS) and plated at a concentration of at 0.625×10⁶ cells/mL. Diluted collagen (type 1, rat tail—BD Cat. #354236) (50 ug/ml in 0.02N acetic acid) was used for coating coverslips and plates in about 10 ml (enough to cover them) at room temperature for one hour. The collagen solution was then removed and rinsed once with PBS. After the cells attached (<18 hrs), the HPM was replaced by Hepatocyte Media (500 mL DMEM high glucose; 30 mg L-methionine; 104 mg L-leucine; 33.72 mg L-ornithine; 200 uL of 5 mM stock dexamethasone; 3 mg Insulin) The HCV infected patient serum was provided by Dr. Chojkier.

### Mouse Primary Hepatocyte Cultures

Primary mouse hepatocytes were obtained as described (7; 8). All hepatocyte manipulations were performed under sterile conditions in a biosafety cabinet. Hepatocytes were isolated by a modified perfusion technique introduced by Seglen (63). A liver was perfused with calcium-free HBSS supplemented with 0.5 mM EGTA for 20 to 30 min and then with 0.05% collagenase [Sigma] dissolved in L-15 medium (with calcium) at 37° C. until the tissue was fully digested. The digested liver was removed, immediately cooled with ice-cold L-15 medium and the cell suspension was strained through serial progressively smaller stainless steel sieves, with a final filtration through 100-micron and 60-micron nylon mesh. The filtered cell suspension was aliquoted into 250-ml tubes and centrifuged three times at 40 g for 3 min at 4° C.

Cells were re-suspended in Hepatocyte Plating Media (500 mL DMEM high glucose; 20% FBS) and plated at a concentration of at 0.625 10⁶ cells/mL. Diluted collagen (type 1, rat tail—BD Cat. #354236) (50 ug/ml in 0.02N acetic acid) was used for coating coverslips and plates in about 10 ml (enough to cover them) at room temperature for one hour. The collagen solution was then removed and rinsed once with PBS. After the cells attached (<18 hrs), the HPM was replaced by Hepatocyte Media (500 mL DMEM high glucose; 30 mg L-methionine; 104 mg L-leucine; 33.72 mg L-ornithine; 200 uL of 5 mM stock dexamethasone; 3 mg Insulin.

### Huh-7 HCV Infection Methods

All of the reagents for the Huh-7 HCV infection system were provided. Viral RNA was in vitro transcribed from the pUC-vJFH cDNA vector linearized with Eco RI as described in the commercial protocol (mMESSAGE mMACHINE Kit, Ambion). AGFP RNA identically in vitro transcribed was used as a positive control for RNA transfection. RNA was transfected into Huh-7.5.1 cells (1×10⁷ cells/ml) with the BioRad Gene Pulser (model:1652076). 10 μg of RNA was added to 400 μl of cells and transferred into an electroporation cuvette with a gap of 0.4 cm. The sample was 0.27 kV, 100 Ohms and 960 μF. Cells were immediately transferred into 30 mls of complete growth media (DMEM, 10% FBS, antibioties (Pen/Strep/GLu), 100 mM Hepes, 1× nonessential amino acids) and plated into 3 T75 tissue culture flasks. Upon confluency, the supernatant was saved, as it contained the infectious virus, and the cells were split 1:4. This was repeated until day 20 post-transfection. Day 18 was associated with peak virus production. Serial fold dilutions were made of the supernatant and put onto Huh-7.5.1 cells. Foci were counted by immuno-staining with antibody to HCV core. Infectivity titers were calculated as the highest dilution of the sample that still retains infectivity. Huh-7.5.1 cells were inoculated with 4×10⁴ ffu (foci forming units)/3×10⁶ cells. Infection occurred within 5 hours of inoculation at 37° C. Infection was measured by RT-PCR with primer sequences 5′-TCTGCGGAACCGGTGAGTA-3′(sense) (SEQ ID NO:37) and 5′-TCAGGCAGTACCACAAGGC-3′ (anti-sense) (SEQ ID NO:38) based on the JFH-1 sequence (Genbank AB047639). The primers allowed for a two temperature PCR with denaturation at 95° C. (30 seconds) and annealing/elongation at 60° C. for 1 minute.

### DNA and Protein Transfection

Cells were cultured as described above and transfected with lipofectamine (GIBCO) for DNA vectors or with the Chariot reagent (Active Motif) for recombinant proteins (1 μg) as described (8) Transfected or expressed proteins were visualized using antibodies specific for HA or His tags or to the protein of interest as described by the PI (8)

### Micro-Injection

Micro-injection of antibodies to HCV E2 was performed at the UCSD Cancer Center Core Microscopy Center (where the PI is a full member), on a re-charge basis.

### Immunoprecipitation, Immunoblotting

HCV E2 (antibodies from BioDesign, Abeam, and two custom antibodies from Pacific Immunology), cyclin G (antibody from Santa Cruz Biotechnology), HSC 70 (antibody from Santa Cruz Biotechnology), clathrin HC (antibody from Santa Cruz Biotechnology), AP50 (antibody from BD Transduction Laboratories and a custom antibody from Pacific Immunology), PIP₂ (antibody from Abeam), PI3K (antibody from Santa Cruz Biotechnology), PDK1 (antibody from Cell signaling), Akt (antibody from Santa Cruz Biotechnology), BAD (antibody from Santa Cruz Biotechnology), pPI3K (antibody from Cell signaling), pPDK1 (antibody from Cell signaling), pAKT (antibody from Cell signaling), pBAD (antibody from Santa Cruz Biotechnology), were detected by immunoblotting the immunoprecipitates from hepatocyte lysates (12) following the chemiluminescence protocol (DuPont) and using purified IgG antibodies conjugated to HRP as described (68). These immunoblots were visualized and recorded by a Kodak 4000×M imaging system.

### Confocal Microscopy

Fluorescent labels were observed using a triple-channel fluorescence microscope or a laser scanning confocal microscope. Fluorochromes utilized included TOPRO-3 (blue) (Molecular Probes, Invitrogen), Alexa 488 (green) (Molecular Probes, Invitrogen) and Alexa 594 (red) (Molecular Probes, Invitrogen) conjugated to secondary antibodies. The primary antibodies were goat anti-HCV E2, mouse anti-cyclin G, mouse anti-HSC70, rabbit anti-clathrin HC, mouse anti-AP50, and rabbit anti-PCNA. The antibody to phosphor-threo156 AP50 was provided. Immuno-staining and analysis were conducted as previously described (8) (61). At least 100 cells were analyzed per experimental point (9). The nuclear morphology was analyzed by staining cells with TOPRO-3 (R&D Systems).

### [³H]-Thymidine Incorporation

Cells were transfected either lipofectamine (GIBCO) for DNA or with Chariot (Active Motif #30100). Transfection reagent was removed and 2 ml/well media was added and incubated at 37° C. for 2 hours. Either EGF (upstate cat #01-101) at 25 ng/ml or TGFα (EMD cat. #PF008) at 25 ng/ml was added as positive controls. EGF inhibitor, PD153035, (Calbiochem #234490) was added to some samples to ensure that DNA replication was due to E2 independently of EGF. 1 μci/ml Thymidine, [methyl-³H] (Perkin Elmer Cat #NET027Z) was added to cells and they were incubated at 37° C. for 48 hours. Media was removed and the cells were washed 2× with ice cold PBS. 0.5 ml of cold 10% Trichloroacetic acid (TCA) was added and incubated at room temperature for 1 hour. TCA was removed and cells were rinsed with ethanol. Cells were harvested in 0.5 ml of 0.1 M NaOH containing 1% SDS. Radioactivity was determined using a Beckman LS6500 liquid scintillation counter.

### Brdu Incorporation

Measurement of cell proliferation was analyzed by DNA incorporation of the thymidine analog 5′-bromo-2′deoxyuridine (Brdu) as described by the manufacturer, Sigma-Aldrich. After cells were synchronized by serum starvation for 24 hours, they were infected with HCV. Media containing 10 mM Brdu was added 12 hours later and incubated for an additional 24 hours. Cells were fixed with 4% paraformaldehyde in PBS and permiabilized with 2% Triton X-100 in PBS. Immuno-staining with anti-Brdu antibody (Sigma-Aldrich) were as previously described (8) (61). The percentage of Brdu cells was counted by fluorescence microscopy or cells were sorted by the flow cytometry core at the Veteran's Medical Center.

### Flow Cytometry

Cells were transfected either lipofectamine (GIBCO) for DNA or with Chariot (Active Motif #30100) with HCV E2 or mutants together with GFP. The cells were trypsinized and suspended in tissue culture medium and stained with Hoechst 33342 (2 μg/ml). They were incubated for 20 minutes at 37° C. Flow cytometry was performed at the flow cytometry core at the Veterans' Medical Center. The cells were sorted according to their DNA content (UV excitation at 340 to 380 nm) and positive transfection (GFP).

### Mutagenesis

The HCV E2 protein was mutated using specific primers with Stratagene's Quick Change site-directed mutagenesis kit as described previously by the PI (8). These mutations were evaluated in vitro kinase assays, in tissue culture transfections and in the Huh-7 HCV infection system.

### Expression and Purification of Recombinant Wild Type and Mutated Proteins

Expression plasmids encoding a given protein were constructed in the T7 expression vector pET3b, as described (12; 17; 32; 68; 69). Bacterial extracts were prepared from bacteria (BL 21/DE-3/pLysS) grown for 4-5 h in the presence of 0/5 mM IPTG, as previously described. (42) Recombinant proteins were purified from these lysates by fractionation on heparin-agarose columns, as described previously (11; 12; 17; 32; 68; 69). Other expression vectors utilized require affinity purification of the recombinant protein through affinity columns, by the methods described by the manufacturer. Affinity purification of nuclear proteins using the cognate DNA binding sequence was performed as described. (41) The purification and identification of deleted proteins were facilitated by the use of specific antibodies against the co-expressed HA or His tags as previously described (8).

### Phosphorylation of AP50 In Vivo and In Vitro

AP50 phosphorylation on Thr¹⁵⁶ was determined in livers of control and HCV-infected livers, in mouse hepatocytes expressing or not E2, and in HCV-infected and control human hepatocytes [treated or not with the AP50 peptides] by confocal microscopy using specific antibodies as described above. Specific antibodies against phosphorylated AP50 Thr¹⁵⁶ were provided (66; 73). As an alternative approach, AP50 was purified by immunoprecipitation, gel electrophoresis and/or HPLC and phosphorylation on Thr¹⁵⁶ were determined by Mass Spectroscopy at the Core Facility, Scripps Research Institute, La Jolla.

AP50 was immunopurified from untransfected primary hepatocytes and subjected to heat inactivation of any associated kinases. Recombinant wild type or mutated E2 were combined with AP50 in the presence of ³²P ATP (MP Biomedicals cat. #35020) and kinase buffer (50 mM Tris-HCL, pH 7.5, 5 mM MgCl₂). The reaction was incubated at room temperature for 1 hour, and run on an SDSPAGE, transferred to a membrane and exposed to film overnight, as described previously (8; 57; 63).

### Affinity Column Chromatography

Catch and Release affinity columns and protocol (Upstate) were used with HCV E2 antibodies (Biodesign) with non-denaturing buffers as specified by the manufacturer. This method was more efficient and specific in purifying HCV virions than the standard immunoprecipitation techniques. Negative and positive control samples were run in parallel.

### Transferrin Internalization Assays

Radioisotopes, Transferrin (human) [¹²⁵I]-diferric (Cat #NEX212), were purchased from Perkin Elmer. Plate was removed from incubator and put in cold room. 1 μci of ¹²⁵I was immediately added to each well and left in cold room for exactly 30 minutes. ¹²⁵I was removed by washing 2× with PBS. 2 ml/well DME High Glucose was added (Gibco) and cells were incubated at 37° C. for indicated time points. At each time point media was removed and 500 μl of surface bound buffer added (0.5% acetic acid, 0.5M NaCl, in PBS) for 2 minutes at room temperature. Surface bound buffer was removed and put into corresponding and saved for counting as this was the surface bound fraction. Cells were washed with 1× PBS and 500 μl of internal buffer (1% Triton X-100+0.5% SDS in PBS) was added and incubated at 37° C. for 5 minutes. Cells were harvested and radioactivity was determined using a Beckman LS6500 liquid scintillation counter with 5 ml Bio-Safe II counting cocktail.

### Development of Additional Antibodies

Antibodies against AP-50-PhosphoThr¹⁵⁶ were induced in rabbits with the epitope CEEQSQITSQVT (SEQ ID NO:39, Phospho), GQIWRRR (SEQ ID NO:40) linked to keyhole limpet hemocyanin as previously described (8).

### Determination of Cell Toxicity

Toxicity of HCV E2 mutants to human hepatocyte cultures was determined by measuring lactic dehydrogenase (LDH) (Sigma) and alanine aminotransferase (ALT) (Weiner Laboratories) in the medium. Positive (Jo2 Ab) and negative (untreated cells) control samples were determined in parallel. LDH assays of culture media were measurements of cellular leakage that indicates cell injury. ALT was enriched in hepatocytes and it's presence in serum or cell culture media was a classic indicator of hepatocyte injury. Indeed, it is the FDA's gold standard for hepatocellular toxicity.

### Statistical Analysis

Results were expressed as mean (±SEM) of at least triplicates unless stated otherwise. Either the Student-t or the Fisher's exact test was used to evaluate the differences of the means between groups, with a P value of <0.05 as significant.

### LITERATURE CITED

1. Hepatitis C: global prevalence. Wkly Epidemiol Rec 72: 341-344, 1997.
2. Alter H J and Seeff L B. Recovery, persistence and sequelae in hepatitis C virus infection: a perspective on the long-term outcome. Semin Liv Dis 20: 17-25, 2000.
3. Alter M J. Epidemiology of hepatitis C. Hepatology 26: 62S-65S, 1997.
4. Blight K J, Grakoui A, Hanson H L and Rice C M. The molecular biology of hepatitis C virus. In: Hepatitis viruses, edited by Ou J-H J. Boston: Kluwer Academic Publishers, 2002, p. 81-108.
5. Branda M and Wands J R. Signal transduction cascades and hepatitis B and C related hepatocellular carcinoma. Hepatology 43: 891-902, 2006.
6. Bruix J, Boix L, Sala M and Llovet J M. Focus on hepatocellular carcinoma. Cancer Cell 5: 215-219, 2004.
7. Bruix J and Sherman M. Management of hepatocellular carcinoma. Hepatology 42: 1208-1236, 2005.
8. Buck M, Poli V, Hunter T and Chojkier M. C/EBPb phosphorylation by RSK creates a functional XEXD caspase inhibitory box critical for cell survival. Mol Cell 8: 807-816, 2001.
9. Buck M, Poli V, van der Geer P, Chojkier M and Hunter T. Phosphorylation of rat serine 105 or mouse threonine 217 in C/EBPb is required for hepatocyte proliferation induced by TGFa. Mol Cell 4: 1087-1092, 1999.
10. Buck M, Zhang L, Hunter T and Chojkier M. Nuclear export of phosphorylated C/EBPb mediates the inhibition of albumin expression by TNFa. EMBO J 20: 6712-6723, 2001.
11. Buck M and Chojkier M. Muscle wasting and dedifferentiation induced by oxidative stress in a murine model of cachexia is prevented by inhibitors of nitric oxide synthesis and antioxidants. EMBO J15: 1753-1765, 1996.
12. Buck M, Turler H and Chojkier M. LAP (NF-IL6), a tissue-specific transcriptional activator, is an inhibitor of hepatoma cell proliferation. EMBO J 13: 851-860, 1994.
13. Camaschella C, Roetto A, Cali A, De Gobbi M, Garozzo G, Carella M, Majorano N, Totaro A and Gasparini P. The gene TFR2 is mutated in a new type of haemochromatosis mapping to 7q22. Nat Genet 25: 14-15, 2000.
14. Cantley L C. The Phosphoinositide 3-Kinase Pathway. Science 296: 1655-1657, 2002.
15. Cope M J, Yang S, Shang C and Drubin D G. Novel protein kinases Ark1p and Prk1p associate with and regulate the cortical actin cytoskeleton in budding yeast. J Cell Biol 144: 1203-1218, 1999.
16. Davila J A, Morgan R O, Shaib Y, McGlynn K A and El-Serag H B. Hepatitis C infection and the increasing incidence of hepatocellular carcinoma: A population-based study. Gastroenterology 127: 1372-1380, 2004.
17. Descombes P, Chojkier M, Lichtsteiner S, Falvey E and Schibler U. LAP, a novel member of the C/EBP gene family, encodes a liver-enriched transcriptional activator protein. Genes Dev 4: 1541-1551, 1990.
18. El-Serag H B. Hepatocellular carcinoma and hepatitis C in the United States. Hepatology 36: S74-S83, 2002.
19. El-Serag H B, Kunik M, Richardson P and Rabeneck L. Psychiatric disorders among veterans with hepatitis C infection. Gastroenterology 123: 476-482, 2002.
20. El-Serag H B and Mason A C. Rising incidence of hepatocellular carcinoma in the United States. N Engl J Med 340: 745-750, 1999.
21. Farazi P A and Depinho R A. Hepatocellular carcinoma pathogenesis: from genes to environment. Nat Rev Cancer 6: 674-687, 2006.
22. Farinati F, Cardin R, De M N, Della L G, Marafin C, Lecis E, Burra P, Floreani A, Cecchetto A and Naccarato R. Iron storage, lipid peroxidation and glutathione turnover in chronic anti-HCV positive hepatitis. J Hepatol 22: 449-456, 1995.
23. Furutani T, Hino K, Okuda M, Gondo T, Nishina S, Kitase A, Korenaga M, Xiao S Y, Weinman S A, Lemon S M, Sakaida I and Okita K. Hepatic iron overload induces hepatocellular carcinoma in transgenic mice expressing the hepatitis C virus polyprotein. Gastroenterology 130: 2087-2098, 2006.
24. Gerotto M, Dal P F, Bortoletto G, Ferrari A, Pistis R, Sebastiani G, Fagiuoli S, Realdon S and Alberti A. Hepatitis C minimal residual viremia (MRV) detected by TMA at the end of Peg-IFN plus ribavirin therapy predicts post-treatment relapse. J Hepatol 44: 83-87, 2006.
25. Greener T, Grant B, Zhang Y, Wu X, Greene L E, Hirsh D and Eisenberg E. Caenorhabditis elegans auxilin: a J-domain protein essential for clathrin-mediated endocytosis in vivo. Nat Cell Biol 3: 215-219, 2001.
26. Hanks S K, Quinn A M and Hunter T. The protein kinase family: conserved features and deduced phylogeny of the catalytic domains. Science 241: 42-52, 1988.
27. Hardie G and Hanks S. The protein kinase factsbook. London; San Diego: Academic Pres, c1995, 1995.
28. Hassan M M, Hwang L Y, Hatten C J, Swaim M, Li D, Abbruzzese J L, Beasley P and Patt Y Z. Risk factors for hepatocellular carcinoma: synergism of alcohol with viral hepatitis and diabetes mellitus. Hepatology 36: 1206-1213, 2002.
29. Heller T, Saito S, Auerbach J, Williams T, Moreen T R, Jazwinski A, Cruz B, Jeurkar N, Sapp R, Luo G and Liang T J. An in vitro model of hepatitis C virion production. Proc Natl Acad Sci USA 102: 2579-2583, 2005.
30. Hentze M W, Muckenthaler M U and Andrews N C. Balancing acts: molecular control of mammalian iron metabolism. Cell 117: 285-297, 2004.
31. Honing S, Ricotta D, Krauss M, Spate K, Spolaore B, Motley A, Robinson M, Robinson C, Haucke V and Owen D J. Phosphatidylinositol-(4,5)-Bisphosphate Regulates Sorting Signal Recognition by the Clathrin-Associated Adaptor Complex AP2. Molecular Cell 18: 519-531, 2005.
32. Houglum K, Buck M, Adir V and Chojkier M. LAP (NF-IL6) transactivates the collagen a₁(1) gene from a 5′ regulatory region. J Clin Invest 94: 808-814, 1994.
33. Houglum K, Buck M and Chojkier M. Tumor necrosis factor a inhibits liver collagen a1(I) gene expression through a tissue-specific regulatory region. Am J Physiol 274: G840-G847, 1998.
34. Houglum K, Lee K S and Chojkier M. Proliferation of hepatic stellate cells is inhibited by phosphorylation of CREB on Serine 133. J Clin Invest 99: 1322-1389, 1997.
35. Ikeda K, Saitoh S, Suzuki Y, Kobayashi M, Tsubota A, Koida I, Arase Y, Fukuda M, Chayama K, Murashima N and Kumada H. Disease progression and hepatocellular carcinogenesis in patients with chronic viral hepatitis: a prospective observation of 2215 patients. J Hepatol 28: 930-938, 1998.
36. Jhappan C, Stahle C, Harkins R N, Fausto N, Smith G H and Merlino G T. TGF alpha overexpression in transgenic mice induces liver neoplasia and abnormal development of the mammary gland and pancreas. Cell 61: 1137-1146, 1990.
37. Johnson S A and Hunter T. Kinomics: methods for deciphering the kinome. Nat Methods 2: 17-25, 2005.
38. Kageyama F, Kobayashi Y, Kawasaki T, Toyokuni S, Uchida K and Nakamura H. Successful interferon therapy reverses enhanced hepatic iron accumulation and lipid peroxidation in chronic hepatitis C. Am J Gastroenterol 95: 1041-1050, 2000.
39. Kanaoka Y, Kimura S H, Okazaki I, Ikeda M and Nojima H. GAK: a cyclin G associated kinase contains a tensin/auxilin-like domain. FEBS Letters 402: 73-80, 1997.
40. Kannan N and Neuwald A F. Evolutionary constraints associated with functional specificity of the CMGC protein kinases MAPK, CDK, GSK, SRPK, DYRK, and CK2alpha. Protein Sci 13: 2059-2077, 2004.
41. Lichtsteiner S and Schibler U. A glycosylated liver-specific transcription factor stimulates transcription of the albumin gene. Cell 57: 1179-1187, 1989.
42. Lichtsteiner S, Wuarin J and Schibler U. The interplay of DNA-binding proteins on the promoter of the mouse albumin gene. Cell 51: 963-973, 1987.
43. Lindenbach B D and Rice C M. Flaviviridae: the viruses and their replication. In: Fields virology, edited by Knipe D M and Howley P M. Philadelphia: Lippincott-Raven, 2001, p. 991-1041.
44. Lindenbach B D, Evans M J, Syder A J, Wolk B, Tellinghuisen T L, Liu C C, Maruyama T, Hynes R O, Burton D R, McKeating J A and Rice C M. Complete Replication of Hepatitis C Virus in Cell Culture. Science 1114016, 2005.
45. Mazzaferro V, Regalia E, Doci R, Andreola S, Pulvirenti A, Bozzetti F, Montalto F, Ammatuna M, Morabito A and Gennari L. Liver transplantation for the treatment of small hepatocellular carcinomas in patients with cirrhosis. N Engl J Med 334: 693-699, 1996.
46. McLaughlin S and Murray D. Plasma membrane phosphoinositide organization by protein electrostatics. Nature 438: 605-611, 2005.
47. Moriya K, Fujie H, Shintani Y, Yotsuyanagi H, Tsutsumi T, Ishibashi K, Matsuura Y, Kimura S, Miyamura T and Koike K. The core protein of hepatitis C virus induces hepatocellular carcinoma in transgenic mice. Nat Med 4: 1065-1067, 1998.
48. Motley A, Bright N A, Seaman M N and Robinson M S. Clathrin-mediated endocytosis in AP-2-depleted cells. J Cell Biol 162: 909-918, 2003.
49. Nakanishi K, Sakamoto M, Yamasaki S, Todo S and Hirohashi S. Akt phosphorylation is a risk factor for early disease recurrence and poor prognosis in hepatocellular carcinoma. Cancer 103: 307-312, 2005.
50. Olusanya O, Andrews P D, Swedlow J R and Smythe E. Phosphorylation of threonine 156 of the mu2 subunit of the AP2 complex is essential for endocytosis in vitro and in vivo. Curr Biol 11: 896-900, 2001.
51. Paolo G D, Moskowitz H S, Gipson K, Wenk M R, Voronov S, Obayashi M, Flavell R, Fitzsimonds R M, Ryan T A and Camilli P D. impaired PtdIns(4,5)P2 synthesis in nerve terminals produces defects in synaptic vesicle trafficking. Nature 431: 415-422, 2004.
52. Pileri P, Uematsu Y, Campagnoli S, Galli G, Falugi F, Petracca R, Weiner A J, Houghton M, Rosa D, Grandi G and Abrignani S. Binding of hepatitis C virus to CD81. Science 282: 938-941, 1998.
53. Pishvaee B, Costaguta G, Yeung B G, Ryazantsev S, Greener T, Greene L E, Eisenberg E, McCaffery J M and Payne G S. A yeast DNA J protein required for uncoating of clathrin-coated vesicles in vivo. Nat Cell Biol 2: 958-963, 2000.
54. Reed K E, Gorbalenya A E and Rice C M. The NS5A/NS5 proteins of viruses from three genera of the family Flaviviridae are phosphorylated by associated serine/threonine kinases. J Virol 72: 6199-6206, 1998.
55. Report 04-5491. Liver Cancer. In: Action Plan for Liver Disease Research, U.S. Department of Health and Human Services: NIH, 2004, p. 137-143.
56. Report 04-5491. Viral Hepatitis. In: Action Plan for Liver Disease Research, U.S. Department of Health and Human Services: NIH, 2004, p. 61-69.
57. Ricotta D, Conner S D, Schmid S L, von Figura K and Honing S. Phosphorylation of the AP2 {micro} subunit by AAK1 mediates high affinity binding to membrane protein sorting signals. The Journal of Cell Biology 156: 791-795, 2002.
58. Roberts L R and Gores G J. Hepatocellular carcinoma: molecular pathways and new therapeutic targets. Semin Liver Dis 25: 212-225, 2005.
59. Robertson B, Myers G, Howard C, Brettin T, Bukh J, Gaschen B, Gojobori T, Maertens G, Mizokami M, Nainan O, Netesov S, Nishioka K, Shin i T, Simmonds P, Smith D, Stuyver L and Weiner A. Classification, nomenclature, and database development for hepatitis C virus (HCV) and related viruses: proposals for standardization. Arch Virol 143: 2493-2503, 1998.
60. Rohde G, Wenzel D and Haucke V. A phosphatidylinositol (4,5)-bisphosphate binding site within {micro}2-adaptin regulates clathrin-mediated endocytosis. The Journal of Cell Biology 158: 209-214, 2002.
61. Rudel T and Bokoch G. Membrane and morphological changes in apoptotic cells regulated by caspase-mediated activation of PAK2. Science 276: 1571-1574, 1997.
62. Sandgren E P, Luetteke N C, Qiu T H, Palmiter R D, Brinster R L and Lee D C. Transforming growth factor alpha dramatically enhances oncogene-induced carcinogenesis in the transgenic mouse pancreas and liver. Mol Cell Biol 13: 320-330, 1993.
63. Seglen P O. Preparation of isolated rat liver cells. Methods Cell Biol 13: 29-83, 1976.
64. Seipp S, Mueller H M, Pfaff E, Stremmel W, Theilmann L and Goeser T. Establishment of persistent hepatitis C virus infection and replication in vitro. J Gen Virol 78: 2467-2476, 1997.
65. Shimotohno K. Hepatitis C virus and its pathogenesis. Semin Cancer Biol 10: 233-240, 2000.
66. Smythe E. Cell biology: light on pits. Nature 431: 641-642, 2004.
67. Smythe E and Ayscough K R. The Ark1/Prk1 family of protein kinases. Regulators of endocytosis and the actin skeleton. EMBO Rep 4: 246-251, 2003.
68. Trautwein C, Caelles C, van der Geer P, Hunter T, Karin M and Chojkier M. Transactivation by NF-IL6/LAP is enhanced by phosphorylation of its activation domain. Nature 364: 544-547, 1993.
69. Trautwein C, van der Geer P, Karin M, Hunter T and Chojkier M. Protein kinase A and C site-specific phosphorylations of LAP (NF-IL6) modulate its binding affinity to DNA-recognition elements. J Clin Invest 93: 2554-2561, 1994.
70. Vivanco I and Sawyers C L. The phosphatidylinositol 3-Kinase AKT pathway in human cancer. Nat Rev Cancer 2: 489-501, 2002.
71. Wakita T, Pietschmann T, Kato T, Date T, Miyamoto M, Zhao Z, Murthy K, Habermann A, Krausslich H G, Mizokami M, Bartenschlager R and Liang T J. Production of infectious hepatitis C virus in tissue culture from a cloned viral genome. Nat Med 2005.
72. Wenk M R and De Camilli P. Inaugural Article: Protein-lipid interactions and phosphoinositide metabolism in membrane traffic: Insights from vesicle recycling in nerve terminals. PNAS 101: 8262-8269, 2004.
73. Zhang C X, Engqvist-Goldstein A E Y, Carreno S, Owen D J, Smythe E and Drubin D G. Multiple Roles for Cyclin G-Associated Kinase in Clathrin-Mediated Sorting Events. Traffic 6: 1103-1113, 2005.
74. Zhang L, Gjoerup O and Roberts T M. The serine/threonine kinase cyclin G-associated kinase regulates epidermal growth factor receptor signaling. PNAS 101: 10296-10301, 2004.
75. Zhong J, Gastaminza P, Cheng G, Kapadia S, Kato T, Burton D R, Wieland S F, Uprichard S L, Wakita T and Chisari F V. Robust hepatitis C virus infection in vitro. Proc Natl Aced Sci USA 102: 9294-9299, 2005.

What is claimed is:

1. An isolated HCV E2 kinase phospho-peptide comprising an immunogenic fragment of a HCV E2 kinase motif.

2. The isolated HCV E2 kinase phospho-peptide of claim 1, wherein said phospho-peptide comprises an amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, immunogenic fragments, or homologs thereof.

3. The isolated HCV E2 kinase phospho-peptide of claim 2, wherein said peptide comprises an amino acid sequence of SEQ ID NO:14, immunogenic fragment, or homologs thereof.

4. The isolated HCV E2 kinase phospho-peptide of claim 1, wherein said HCV E2 kinase motif comprises an amino acid sequence as set forth in SEQ ID NO:21, SEQ ID NO:22, SEQ ID NO:23, SEQ ID NO:24, SEQ ID NO:25, SEQ ID NO:25, SEQ ID NO:26, SEQ ID NO:27, SEQ ID NO:28, SEQ ID NO:29, SEQ ID NO:30, SEQ ID NO:31, SEQ ID NO:32, immunogenic fragments, or homologs thereof.

5. The isolated HCV E2 kinase phospho-peptide of claim 1, wherein said HCV E2 motif comprises an amino acid sequence as set forth in SEQ ID NO:33 or homologs thereof.

6. The isolated HCV E2 kinase phospho-peptide of claim 1, wherein said HCV E2 motif comprises an amino acid sequence as set forth in SEQ ID NO:34, or homologs thereof.

7. An antibody for HCV immunotherapy which is cross-reactive with said isolated HCV E2 kinase phospho-peptide of claim 1.

8. The antibody of claim 7, wherein said antibody is cross-reactive with an unphosphorylated motif of an amino acid sequence of SEQ ID NO:14.

9. The antibody of claim 7, wherein said antibody is cross-reactive with a phosphorylated motif of an amino acid sequence of SEQ ID NO:14.

10. The antibody of claim 9, wherein said phosphorylated motif comprises an amino acid tyrosine (Y).

11. The antibody of claim 7, wherein said antibody is cross-reactive with said immunogenic fragment of said isolated HCV E2 phospho-peptide.

12. The antibody of claim 11, wherein said immunogenic fragment comprises an amino acid sequence as set forth in SEQ ID NO:33.

13. The antibody of claim 11, wherein said immunogenic fragment comprises an amino acid sequence as set forth in SEQ ID NO:34.

14. A pharmaceutical composition comprising said isolated HCV E2 phospho-peptide of any one of claims 1-6, and a pharmaceutically acceptable carrier.

15. A pharmaceutical composition comprising said antibody of any one of claims 7-13, and a pharmaceutically acceptable carrier.

16. A method to passively immunize against HCV comprising administering to a subject in need an effective amount of one or more antibodies of any one of claims 7-13.

17. A method to passively immunize against HCV comprising administering to a subject in need an effective amount of said pharmaceutical composition of claim 15.

18. A method to actively immunize against HCV comprising administering to a subject in need an effective amount of one or more isolated HCV E2 phospho-peptide of any one of claims 1-6.

19. A method to actively immunize against HCV comprising administering to a subject in need an effective amount of said pharmaceutical composition of claim 14.


SEQUENCE LISTING (124 sequences)

SEQ ID NO: 1 - protein, 10 aa, Hepatitis C virus
ETHVTGGSAR

SEQ ID NO: 2 - protein, 14 aa, Hepatitis C virus
TTAGLVGLLTPGAK

SEQ ID NO: 3 - protein, 38 aa, Hepatitis C virus
QNIQLINTNGSWHTNSTALNCNESLNTGWLAGLFYQHK

SEQ ID NO: 4 - protein, 9 aa, Hepatitis C virus
FNSSGCPER

SEQ ID NO: 5 - protein, 5 aa, Hepatitis C virus
LASCR

SEQ ID NO: 6 - protein, 22 aa, Hepatitis C virus
LTDFAQGWGPISYANGSGLDER

SEQ ID NO: 7 - protein, 9 aa, Hepatitis C virus
PYCWHYPPR

SEQ ID NO: 8 - protein, 21 aa, Hepatitis C virus
SVCGPVYCFTPSPVVVGTTDR

SEQ ID NO: 9 - protein, 22 aa, Hepatitis C virus
SGAPTYSWGANDTDVFVLNNTR

SEQ ID NO: 10 - protein, 19 aa, Hepatitis C virus
PPLGNWFGCTWMNSTGFTK

SEQ ID NO: 11 - protein, 25 aa, Hepatitis C virus
VCGAPPCVIGGVGNNTLLCPTDCFR

SEQ ID NO: 12 - protein, 8 aa, Hepatitis C virus
HPEATYSR

SEQ ID NO: 13 - protein, 10 aa, Hepatitis C virus
CGSGPWITPR

SEQ ID NO: 14 - protein, 8 aa, Hepatitis C virus
CMVDYPYR

SEQ ID NO: 15 - protein, 14 aa, Hepatitis C virus
LWHYPCTINYTIFK

SEQ ID NO: 16 - protein, 9 aa, Hepatitis C virus
MYVGGVEHR

SEQ ID NO: 17 - protein, 9 aa, Hepatitis C virus
LEAACNWTR

SEQ ID NO: 18 - protein, 55 aa, Hepatitis C virus
SELSPLLLSTTQWQVLPCSFTTLPALSTGLIHLHQNIVDQYLYGVGSSIASWAIK

SEQ ID NO: 19 - protein, 15 aa, Hepatitis C virus
WEYVVLLFLLLADAR

SEQ ID NO: 20 - protein, 16 aa, Hepatitis C virus
VCSCLWMMLLISQAEA

SEQ ID NO: 21 - protein, 8 aa, Hepatitis C virus
VTGGSAGR

SEQ ID NO: 22 - protein, 7 aa, Hepatitis C virus
PGQAKQN

SEQ ID NO: 23 - protein, 5 aa, Hepatitis C virus
ERLAS

SEQ ID NO: 24 - protein, 7 aa, Hepatitis C virus
HYPPRPC

SEQ ID NO: 25 - protein, 7 aa, Hepatitis C virus
CGPVYCF

SEQ ID NO: 26 - protein, 7 aa, Hepatitis C virus
DVFVLNN

SEQ ID NO: 27 - protein, 13 aa, Hepatitis C virus
LCPTDCFRKHPEA

SEQ ID NO: 28 - protein, 9 aa, Hepatitis C virus
FKVRMYVGG

SEQ ID NO: 29 - protein, 12 aa, Hepatitis C virus
AACNWTERGERC

SEQ ID NO: 30 - protein, 18 aa, Hepatitis C virus
DRSELSPLLLSTTQWQVL

SEQ ID NO: 31 - protein, 13 aa, Hepatitis C virus
PCSFTTLPASTGL

SEQ ID NO: 32 - protein, 16 aa, Hepatitis C virus
EYVVLLFLLLADARVC

SEQ ID NO: 33 - protein, 6 aa, Artificial Sequence
  Description of Artificial Sequence Synthetic peptide
ELSPLL

SEQ ID NO: 34 - protein, 23 aa, Artificial Sequence
  Description of Artificial Sequence Synthetic peptide
LSPLLELSPLLELSPLLELSPLL

SEQ ID NO: 35 - protein, 6 aa, Artificial Sequence
  Description of Artificial Sequence Synthetic peptide
XXXXLX

SEQ ID NO: 36 - protein, 20 aa, Artificial Sequence
  Description of Artificial Sequence Synthetic peptide
QGEVQRRRQRRKKRGYGGGG

SEQ ID NO: 37 - DNA, 19 bp, Artificial Sequence
  Description of Artificial Sequence Synthetic primer
tctgcggaaccggtgagta

SEQ ID NO: 38 - DNA, 19 bp, Artificial Sequence
  Description of Artificial Sequence Synthetic primer
tcaggcagtaccacaaggc

SEQ ID NO: 39 - protein, 12 aa, Artificial Sequence
  Description of Artificial Sequence Synthetic peptide
CEEQSQITSQVT

SEQ ID NO: 40 - protein, 7 aa, Artificial Sequence
  Description of Artificial Sequence Synthetic peptide
GQIWRRR

SEQ ID NO: 41 - protein, 7 aa, Artificial Sequence
  Description of Artificial Sequence Synthetic peptide
XXXQXTG

SEQ ID NO: 42 - protein, 14 aa, Artificial Sequence
  Description of Artificial Sequence Synthetic peptide
XXHXDXKPQNXLXX

SEQ ID NO: 43 - protein, 14 aa, Hepatitis C virus
LLTPGAKQNIQLIN

SEQ ID NO: 44 - protein, 14 aa, Hepatitis C virus
LLTPGARQNIQLIN

SEQ ID NO: 45 - protein, 201 aa, Plasmodium farciparum
MSLLQSALDFLAGPGSLGGASGRDQSDFVGQTVELGELRLRVRRVLAEGGFAFVYEAQDVGSGREYALKRLLSNEEEKNRAIIQEVCFMKKLSGHPNIVQFCSAASIGKEESDTGQAEFLLLTELCKGQLVEFLKKMESRGPLSCDTVLKIFYQTCRAVQHMHRQKPPIIHRDLKVENLLLSNQGTIKLCDFGSATTISHY

SEQ ID NO: 46 - protein, 201 aa, Hepatitis C virus
LGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 47 - protein, 201 aa, Hepatitis C virus
  MOD_RES (66)..(66): Phe or Glu
LGNWFGCTWMNSTGFTKVCGAPPCVIGGVGNNTLACPTDCFRKHPEATYSRCGSGPWITPRCMVDXPYRLWHYPCTINYTIFKVRMYVGGVEHRLEAACNWTRGERCDLADRARSELSPLALSTTQWQVAPCSFTTLPALSTGLIHLHQNAVDVQYLYGVGSSIASWAAKWEYVVLLFLALADARVCSCLWMMLLISQAEA

SEQ ID NO: 48 - protein, 8 aa, Caenorhabditis elegans
IPGGKKKE

SEQ ID NO: 49 - protein, 8 aa, Plasmodium farciparum
LIGGKVYN

SEQ ID NO: 50 - protein, 8 aa, Caenorhabditis elegans
AKGGFGTV

SEQ ID NO: 51 - protein, 8 aa, Enterobacteria phage T7
KRQGMLID

SEQ ID NO: 52 - protein, 8 aa, Staphylococcus aureus
DAGGTLIK

SEQ ID NO: 53 - protein, 8 aa, Mycobacterium tuberculosis
GRGGMGEV

SEQ ID NO: 54 - protein, 6 aa, Caenorhabditis elegans
VTLKLD

SEQ ID NO: 55 - protein, 6 aa, Plasmodium farciparum
YMAKDL

SEQ ID NO: 56 - protein, 6 aa, Caenorhabditis elegans
VAVKIM

SEQ ID NO: 57 - protein, 6 aa, Enterobacteria phage T7
TTLKCL

SEQ ID NO: 58 - protein, 6 aa, Staphylococcus aureus
ELTKNI

SEQ ID NO: 59 - protein, 6 aa, Mycobacterium tuberculosis
VAVKLM

SEQ ID NO: 60 - protein, 5 aa, Caenorhabditis elegans
ECMLL

SEQ ID NO: 61 - protein, 5 aa, Plasmodium farciparum
EIHIV

SEQ ID NO: 62 - protein, 5 aa, Caenorhabditis elegans
EIDMM

SEQ ID NO: 63 - protein, 5 aa, Enterobacteria phage T7
EDSGA

SEQ ID NO: 64 - protein, 5 aa, Staphylococcus aureus
EKLCL

SEQ ID NO: 65 - protein, 5 aa, Mycobacterium tuberculosis
EARIA

SEQ ID NO: 66 - protein, 7 aa, Caenorhabditis elegans
HKPPHYV

SEQ ID NO: 67 - protein, 7 aa, Plasmodium farciparum
HTDLIEV

SEQ ID NO: 68 - protein, 7 aa, Caenorhabditis elegans
HENIIQL

SEQ ID NO: 69 - protein, 7 aa, Enterobacteria phage T7
HYKKAEI

SEQ ID NO: 70 - protein, 7 aa, Staphylococcus aureus
HDLADYI

SEQ ID NO: 71 - protein, 7 aa, Mycobacterium tuberculosis
HVVPIHD

SEQ ID NO: 72 - protein, 16 aa, Hepatitis C virus
CGPVYCFTPSPVVVGT

SEQ ID NO: 73 - protein, 16 aa, Caenorhabditis elegans
ILSVEKYGVEYVKSEV

SEQ ID NO: 74 - protein, 16 aa, Plasmodium farciparum
NNFLKFFTTSNLSGDI

SEQ ID NO: 75 - protein, 16 aa, Caenorhabditis elegans
NGNGKLKLCDFGSATT

SEQ ID NO: 76 - protein, 16 aa, Enterobacteria phage T7
GGAFSIDPEELIKEVE

SEQ ID NO: 77 - protein, 16 aa, Staphylococcus aureus
GGMIQGLGYLLSQITD

SEQ ID NO: 78 - protein, 16 aa, Mycobacterium tuberculosis
QMFLEMRLVEGTDSVL

SEQ ID NO: 79 - protein, 21 aa, Hepatitis C virus
PTYSWGANDDTDVFVLNNTRP

SEQ ID NO: 80 - protein, 21 aa, Caenorhabditis elegans
TTMCYRSPEMIDFYSGLEIGL

SEQ ID NO: 81 - protein, 21 aa, Plasmodium farciparum
KEPKDKLENNPDGNKLQNDKD

SEQ ID NO: 82 - protein, 21 aa, Caenorhabditis elegans
YTTPITRSPEVDVYSNWPIGK

SEQ ID NO: 83 - protein, 18 aa, Enterobacteria phage T7
EVARQKEIDRAKARKERH

SEQ ID NO: 84 - protein, 21 aa, Staphylococcus aureus
HIYKDTEPPIPGDLTAANFGH

SEQ ID NO: 85 - protein, 21 aa, Mycobacterium tuberculosis
LTPPRAVAIITQIASALDAAH

SEQ ID NO: 86 - protein, 5 aa, Caenorhabditis elegans
PNIPD

SEQ ID NO: 87 - protein, 13 aa, Plasmodium farciparum
KLQNDSGKLQNEK

SEQ ID NO: 88 - protein, 13 aa, Caenorhabditis elegans
EHPFDGSALAIIN

SEQ ID NO: 89 - protein, 13 aa, Enterobacteria phage T7
LEARRFKRRNRKA

SEQ ID NO: 90 - protein, 13 aa, Staphylococcus aureus
LHHLDADFTPSNK

SEQ ID NO: 91 - protein, 13 aa, Mycobacterium tuberculosis
VMHRDVKPQNILI

SEQ ID NO: 92 - protein, 9 aa, Caenorhabditis elegans
IKVLIYMLL

SEQ ID NO: 93 - protein, 9 aa, Plasmodium farciparum
GKLQNDSGK

SEQ ID NO: 94 - protein, 9 aa, Caenorhabditis elegans
GKYKKPPPV

SEQ ID NO: 95 - protein, 9 aa, Enterobacteria phage T7
HKAKRERNL

SEQ ID NO: 96 - protein, 9 aa, Staphylococcus aureus
FKTENIVYI

SEQ ID NO: 97 - protein, 9 aa, Mycobacterium tuberculosis
AYLVDFGIA

SEQ ID NO: 98 - protein, 11 aa, Hepatitis C virus
AACNWTRGERC

SEQ ID NO: 99 - protein, 11 aa, Caenorhabditis elegans
YQTSVLAFEAN

SEQ ID NO: 100 - protein, 11 aa, Plasmodium farciparum
DNDKKNKSEIS

SEQ ID NO: 101 - protein, 11 aa, Caenorhabditis elegans
KCLTPNEDERI

SEQ ID NO: 102 - protein, 11 aa, Enterobacteria phage T7
QQAAWRWAERQ

SEQ ID NO: 103 - protein, 11 aa, Staphylococcus aureus
NALLRKVVEDY

SEQ ID NO: 104 - protein, 8 aa, Mycobacterium tuberculosis
SATTDEKL

SEQ ID NO: 105 - protein, 18 aa, Caenorhabditis elegans
DAVPSLKSCIQLMRDGSN

SEQ ID NO: 106 - protein, 18 aa, Plasmodium farciparum
DKQNSVPDQSENQSKSHS

SEQ ID NO: 107 - protein, 18 aa, Caenorhabditis elegans
DILDLMNVQPVQAEQSIE

SEQ ID NO: 108 - protein, 18 aa, Enterobacteria phage T7
DVLGRTNNAMLWVNMFSG

SEQ ID NO: 109 - protein, 18 aa, Staphylococcus aureus
KPYYVENGAFSGAIGALY

SEQ ID NO: 110 - protein, 18 aa, Mycobacterium tuberculosis
DIYALACVLHECLTGAPP

SEQ ID NO: 111 - protein, 22 aa, Hepatitis C virus
PCSFTTLPALSTGLIHLHQNIV

SEQ ID NO: 112 - protein, 22 aa, Caenorhabditis elegans
PRNKRDSSPRNPEAPPIQSSSK

SEQ ID NO: 113 - protein, 22 aa, Plasmodium farciparum
YNLIKIQSKDTLIIDHVDRDFP

SEQ ID NO: 114 - protein, 22 aa, Caenorhabditis elegans
SQAAKGFFTMQDKLFSNLTSLK

SEQ ID NO: 115 - protein, 22 aa, Enterobacteria phage T7
DFKALEERIALHWRNADRMAIA

SEQ ID NO: 116 - protein, 3 aa, Staphylococcus aureus
LEK

SEQ ID NO: 117 - protein, 21 aa, Mycobacterium tuberculosis
YRADSAGDLVSSHLMGPIPQP

SEQ ID NO: 118 - protein, 36 aa, Hepatitis C virus
SWAIKWEYVVLLFLLLADARVCSCLWMMLLISQAEA

SEQ ID NO: 119 - protein, 39 aa, Caenorhabditis elegans
VVPNVPSISPVPPVGLPHLRLPSKGSTDETDETDGSQVR

SEQ ID NO: 120 - protein, 39 aa, Plasmodium farciparum
FDVIQNHDLKKKEDVLNINRNNEHDKNAITCSNEYYNLS

SEQ ID NO: 121 - protein, 39 aa, Caenorhabditis elegans
VVQQTNKMGWGMEPTNTTPRPGHPSTSPKLVPKTHRSNQ

SEQ ID NO: 122 - protein, 16 aa, Enterobacteria phage T7
GLTLNIDKQLDAMLMG

SEQ ID NO: 123 - protein, 23 aa, Staphylococcus aureus
TVLRGQKPYYVENGAIGALYLEK

SEQ ID NO: 124 - protein, 39 aa, Mycobacterium tuberculosis
IPKAFDAVVARGMAKKPEDRYASAGDLALAAHEALSDPD